(19) United States
(12) Patent Application Publication
(10) Pub. No.: US 2024/0282403 A1
(43) Pub. Date: Aug. 22, 2024

(54) DETERMINING PHARMACOGENOMICS GENE STAR ALLELES USING HIGH-THROUGHPUT TARGETED GENOTYPING

(71) Applicant: Illumina, Inc., San Diego, CA (US)

(72) Inventors: Julia SOMMER, San Diego, CA (US); Yong LI, San Diego, CA (US)

(73) Assignee: Illumina, Inc., San Diego, CA (US)

(21) Appl. No.: 18/438,627

(22) Filed: Feb. 12, 2024

Related U.S. Application Data
(60) Provisional application No. 63/486,039, filed on Feb. 20, 2023.
(60) Provisional application No. 63/606,075, filed on Dec. 4, 2023.

(51) Int. Cl. G16B 5/20; G06N 7/01; G16B 20/20; G16B 40/20

(52) U.S. Cl. CPC G16B 5/20; G06N 7/01; G16B 20/20; G16B 40/20

(57) ABSTRACT

The determination of pharmacogenomics gene star alleles using high-throughput targeted genotyping includes obtaining input genetic sequence variation data from a high-throughput genotyping platform based on a pharmacogenomic genotyping of a sample, applying a Bayesian graphical model to determine a plurality of different star allele calls corresponding to the sample, and providing a respective quality score for each star allele call of the plurality of different star allele calls. For instance, the application of the Bayesian graphical model uses multi-solution integer programming to explore a model space of the Bayesian graphical model in a first phase that includes structural variant candidate identification and a second phase that includes star allele candidate identification based on the structural variant candidate identification, to determine the plurality of different star allele calls.

### BACKGROUND

Pharmacogenomics (PGx) studies the role of genetic variation in individuals' responses to drug type and dosages with the goal of providing individualized treatment recommendations for better efficacy and reduced side effects. Many genes have been implicated in modulating individuals' drug responses. Examples of PGx genes include:
CYP2D6, G6PD, CYP2C19, CYP2C9, HLA-B, UGT1A1, CACNA1S, RYR1, TPMT, CYP2B6, DPYD, SLCO1B1, CYP3A5, VKORC1, CYP4F2, CFTR, F5, NUDT15, IFNL3/IFNL4, HLA-A, MT-RNR1, and CYP2A6.

Typically, a PGx gene contains one or more Core Variants. Core Variants are usually single nucleotide variants or small indels on the PGx gene that alter the functions of the translated protein. Combinations of Core Variants determine the protein's function, e.g., on drug metabolism. Star-alleles (sometimes written as “star alleles”) are verified combinations or haplotypes of these Core Variants that have been found to be present in a population. Star-alleles can also include structural variations (SVs), including hybridizations with nearby pseudogenes, multiplications, deletions, etc.

### SUMMARY

Shortcomings of the prior art are overcome and additional advantages are provided through the provision of a computer-implemented method for determining pharmacogenomics gene star alleles using high-throughput targeted genotyping. The method obtains input genetic sequence variation data from a high-throughput genotyping platform based on a pharmacogenomic genotyping of a sample, applies a Bayesian graphical model to determine a plurality of different star allele calls corresponding to the sample, and provides a respective quality score for each star allele call of the plurality of different star allele calls.

Further, a computer system is provided that includes a memory and a processor in communication with the memory, wherein the computer system is configured to perform a method for determining pharmacogenomics gene star alleles using high-throughput targeted genotyping. The method obtains input genetic sequence variation data from a high-throughput genotyping platform based on a pharmacogenomic genotyping of a sample, applies a Bayesian graphical model to determine a plurality of different star allele calls corresponding to the sample, and provides a respective quality score for each star allele call of the plurality of different star allele calls.

Yet further, a computer program product including a computer readable storage medium readable by a processing circuit and storing instructions for execution by the processing circuit is provided for performing a method for determining pharmacogenomics gene star alleles using high-throughput targeted genotyping. The method obtains input genetic sequence variation data from a high-throughput genotyping platform based on a pharmacogenomic genotyping of a sample, applies a Bayesian graphical model to determine a plurality of different star allele calls corresponding to the sample, and provides a respective quality score for each star allele call of the plurality of different star allele calls.

In one or more embodiments, the high-throughput genotyping platform includes a microarray-based genotyping platform.

In one or more embodiments, the input genetic sequence variation data includes genotype data and copy number variant call data.

In one or more embodiments, the genotype and copy number data includes B-allele frequency (BAF) and log R ratio data.

In one or more embodiments, the applying the Bayesian graphical model uses multi-solution integer programming to explore a model space of the Bayesian graphical model in (i) a first phase including structural variant (SV) candidate identification and (ii) a second phase including star allele candidate identification based on the SV candidate identification, to determine the plurality of different star allele calls.

In one or more embodiments, the first phase identifies a plurality of SV candidates and evaluates, for each SV candidate of the plurality of SV candidates, a cost of the SV candidate.

In one or more embodiments, the cost of the SV candidate includes a log transformed likelihood.

In one or more embodiments, multiple SV candidates, of the plurality of SV candidates, meeting or exceeding a predefined likelihood threshold are output from the first phase to result in multiple SV candidates provided to the second phase.

In one or more embodiments, a constraint is provided as part of the SV candidate identification to ensure that at least two SV candidates are provided to the second phase.

In one or more embodiments, the second phase identifies a plurality of star allele candidates and evaluates, for each star allele candidate of the plurality of star allele candidates, a cost of the star allele candidate.

In one or more embodiments, the cost of the star allele candidate includes a log transformed likelihood.

In one or more embodiments, each star allele call of the plurality of different star allele calls determined by applying the Bayesian graphical model corresponds to a star allele candidate identified by the second phase and a corresponding SV candidate identified by the first phase, and the respective quality score for the star allele call of the plurality of different star allele calls determined by the applying the Bayesian graphical model includes a composite of (i) the cost of the star allele candidate identified by the second phase and (ii) the cost of the SV candidate identified by the first phase.

In one or more embodiments, the composite includes a sum of the cost of the star allele candidate identified by the second phase and the cost of the SV candidate identified by the first phase.

In one or more embodiments, the Bayesian graphical model considers qualities and population frequencies of structural variants and star alleles in determining the respective quality score for each star allele call of the plurality of different star allele calls.

In one or more embodiments, the method further includes, based on the respective quality score for each star allele call of the plurality of different star allele calls, ranking the plurality of different star allele calls.

In one or more embodiments, the respective quality score for each star allele call of the plurality of different star allele calls includes a log transformed likelihood converted to a posterior probability.

In one or more embodiments, the method further includes providing, for each star allele call of the plurality of different star allele calls, one or more of (i) supporting variants for the star allele call, (ii) missing and/or masked Core Variants, or (iii) missing pharmacogenomic-related variants.

Additional features and advantages are realized through the concepts described herein.

### BRIEF DESCRIPTION OF THE DRAWINGS

Aspects described herein are particularly pointed out and distinctly claimed as examples in the claims at the conclusion of the specification. The foregoing and other objects, features, and advantages of the disclosure are apparent from the following detailed description taken in conjunction with the accompanying drawings in which:

FIG. 1 depicts an example workflow in accordance with aspects described herein;

FIG. 2 depicts an example Bayesian graphical model in accordance with aspects described herein;

FIG. 3 depicts an example sub-network (of FIG. 2) associated with an SV model space, in accordance with aspects described herein;

FIG. 4 depicts examples of SV config probabilities in accordance with aspects described herein;

FIG. 5 depicts example NR probability calculations, in accordance with aspects described herein;

FIG. 6 depicts an example sub-network (of FIG. 2) associated with an Allele model space, in accordance with aspects described herein;

FIGS. 7A-7G depict example results output for candidate solutions, in accordance with aspects described herein;

FIGS. 8A-8B depict additional example results output for candidate solutions, in accordance with aspects described herein;

FIGS. 9A-9C depict example comparison results from testing the StARray approach against a conventional caller;

FIG. 10 depicts an example process for star allele calling, in accordance with aspects described herein; and

FIG. 11 depicts an example of a computer system and associated devices to incorporate and/or use aspects described herein.

### DETAILED DESCRIPTION

For many PGx applications, it is desired to determine the diplotypes of PGx genes, i.e., to determine the star-alleles combinations present in a diploid human genome. Aspects presented herein describe approaches for PGx gene diplotyping. Examples use high throughput targeted genotyping, by way of a high-throughput genotyping platform. In one example, a microarray-based genotyping platform is used, such as the BeadArray technology offered by Illumina, Inc. of San Diego, California. Illumina's BeadArray with Infinium Assay provides the genotype of specific PGx small variant alleles, as well as the copy number calls associated with the small variants or specific target regions of the PGx genes. In a different example, the high-throughput genotyping platform comprises a targeted Next-Generation Sequencing (NGS) platform. Star allele calling methods presented herein enable the determination of diplotypes in a sample from the small variant genotypes, associated B allele frequencies (BAFs), and subgenic copy numbers of a PGx gene of interest.

In examples:
Aspects described herein take array genotyping and copy number variant (CNV) calling as well as raw signal (R and BAF) as input;
Example methods are tolerant to noises as well as genotyping and CNV errors;
Example methods combine structural variant and star-allele determination in one coherent framework;
Example methods provide quality measures associated with determined star-alleles;
Example methods provide ranked alternative star alleles interpretations for samples;
Example methods capture population frequency of variants and star alleles to enable population-specific star allele detection.

The calling of star alleles is a challenging task, especially for complex genetic loci such as the CYP2D6/CYP2D7 locus that has over 140 known CYP2D6 Star-Allele and structural variant configurations in the population. The following steps are traditionally followed to determine star-alleles: (i) Core Variant detection, (ii) SV detection, and (iii) phasing of variants into star-alleles.

Challenges of star-allele calling include high homology between the gene of interest and its pseudogenes, data and platform-specific error patterns, incomplete PGx database information and standardization, accurate SV calling, and phasing of distant Core Variants, as examples.

Arrays present a powerful tool for identifying tens of thousands of PGx biomarkers in thousands of samples in high throughput workflows. Aspects described herein present a tool (which may be referred to herein as StARray) for identifying star-alleles on the array platform. The tool presents several advances to address challenges specific to array data through a novel model-based approach, for instance based on a Bayesian graphical model customized to the array data type. Bayesian graphical models, also referred to as Bayesian networks, are an example type of probabilistic model. Multi-solution integer programming is used to explore the Bayesian graphical model space in two phases: 1) SV solution (i.e., SV candidate) identification; and 2) Allele solution (i.e., star allele candidate) identification, as described herein, to determine and score different possible star allele calls for output.

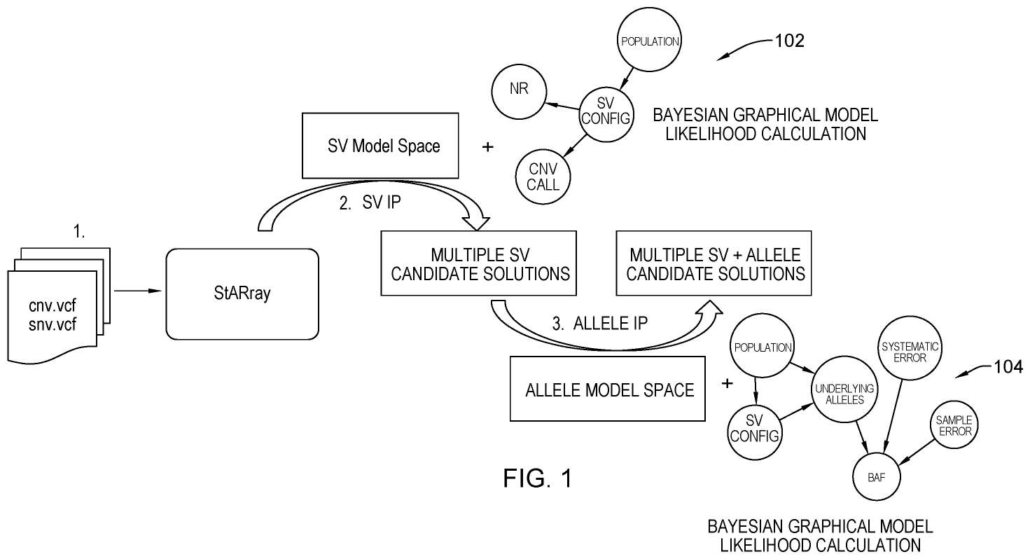

FIG. 1 depicts an example workflow for StARray. The workflow may be performed by one or more computer systems, i.e., by way of one or more process(es) that execute to perform functions. Referring to FIG. 1, a StARray process receives (at 1.) input genetic sequence variation data. In examples, this is by way of one or more variant call format (vcf) data files. As one specific example, the process receives cnv.vcf file(s) and snv.vcf file(s) that are produced by a CNV caller tool. The cnv.vcf file(s) contain information about CNV events, while the snv.vcf file(s) contain information about single nucleotide variant (SNV) and indel events.

Additional aspects of the workflow involve StARray performing integer programming (IP) to explore the array specific Bayesian graphical model space. The model space is decomposed, in accordance with embodiments described herein, into two sub-problems and associated sub-networks (102, 104): 1) SV solution model (for SV candidate identification), and 2) Star allele solution model (for star allele candidate identification). Hence, a StARray process applies (at 2.) SV integer programming (SV IP) to explore the SV model space and evaluates the cost (e.g., as a log transformed likelihood) of each SV solution. The solutions meeting/exceeding a predefined (automatically and/or by a user) likelihood ratio threshold may be a returned/output from the SV IP, resulting in one or multiple SV solutions (also referred to herein as “candidate SV solution” or simply “SV candidate”) to be output to the next phase.

Then, for each of the candidate SV solutions meeting the likelihood ratio threshold (i.e., output of the SV IP), a StARray process utilizes (at 3.) Allele integer programming (allele IP) to explore the constrained allele model space, and evaluates the cost (e.g., log transformed likelihood) of each possible Allele solution (also referred to herein as “star allele candidate”). The likelihood of the entire graphical model is the sum of the likelihoods of these SV and Allele ‘sub-problems’. Thus, the workflow produces one or multiple SV+Allele candidate solutions (calls), each with a respective likelihood determined from the cost/likelihood of the SV sub-problem and the cost/likelihood of the Allele sub-problem. In embodiments discussed herein, “likelihood” and “log likelihood” refer to a negative log-likelihood, also referred to as the logistic loss.

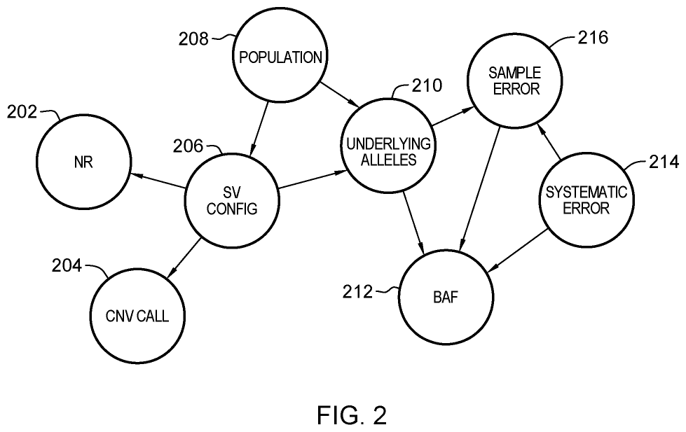

Further details of the workflow presented with generality in FIG. 1 are now discussed. Aspects utilize a platform-specific Bayesian graphical model for array data. FIG. 2 depicts an example such Bayesian graphical model. The graphical model of FIG. 2 is a composite of the models 102 and 104 of FIG. 1.

The likelihood of a particular SV solution found by the SV IP is P(CNV Call|SV)P(NR|SV Config)P(SV Config|Population), explained further below. The likelihood of a particular Allele solution found by the Allele IP (also referred to interchangeably herein as ‘star-allele IP’) is P(Underlying Alleles SV Config, Population)P(BAF|Underlying Alleles, Sample Error, Systematic Error)P(Sample Error Underlying Alleles, Systematic Error)P(Systematic Error), explained further below. These likelihoods are derived from the structure of the graph following principles of Bayesian graphical models.

In graph of FIG. 2, NR 202 is the normalized Log R ratio (LRR) produced by the CNV caller tool for a given gene region. The value CNV Call 204 is the Copy Number call that the CNV caller tool produced for a given gene region. SV Config 206 is a configuration of SV variations found by the SV IP. For example, aspects might return two complete alleles, and 1 hybrid allele as an SV configuration solution. Population 208 is the population used for calculating the SV and Star-allele frequencies. This portion of the Bayesian graphical model is solved by the SV IP.

The Underlying Alleles 210 are the Star-Alleles called by the Allele IP. For example, for a complete CYP2D6 SV, the underlying Star-Alleles might be CYP2D6*2 or CYP2D6*4. The BAF 212 is the B-Allele Frequency of a Core Variant for a Star-Allele in a given Allele solution. Systematic Error 214 (which may be referred to herein also as Systematic_{Error} or Systematic_error) refers to instances where clustering (e.g., GenTrain clustering) error or assay batch effect exists, producing excessive false positive variant calls. StARray can detect such variant level systematic error by comparing sample variant call frequency to known variant population frequency using a normal approximation (as an example). Sample Error 216 is the distance the sample is from the cluster center and is reflected by the GenCall (GS) score. The Allele IP is used to find feasible solutions for this component of the Bayesian graph utilizing feasible SV solutions produced and output by the SV IP.

### SV IP:

The SV Calling uses Integer Programming (IP) to explore the solution space of the Bayesian graphical model to find solution sets of structural variants (SV) corresponding to the input CNV regions from a given cnv.vcf file. One feature of the SV IP is that, unlike other solutions, it can return multiple SV candidates meeting likelihood ratio thresholds. A high-level pseudocode for the SV IP can be found below.

Example pseudocode for SV IP is as follows:

```
sv_vectors, weights = get_sv_vectors_and_weights(input_cnv_vcf)
sv_solution = get_initial_sv_solution(sv_vectors, weights,
input_cnv_vcf)
add_to_previous_solutions(sv_solution, previous_solutions)
if sv_solution.feasible:
  add_to_solution_set(sv_solution, solutions_set)
sv_solution.cost =
get_solution_likelihood_from_Bayesian_graphical_model( )
mle = sv_solution.cost
while sv_solution.feasible and number_of_sv_solutions <= 10:
  sv_solution = get_sv_solution(sv_vectors, weights, input_cnv_vcf,
  previous_solutions)
  sv_solution.cost =
  get_solution_liklihood_from_bayesian_graphical_model( )
  if sv_solution.cost < mle:
    mle = sv_solution.cost
  if sv_solution.cost/mle >= min_log_ratio and sv_solution.feasible:
    add_to_solution_set(sv_solution, solutions_set)
  add_to_previous_solutions(sv_solution, previous_solutions)
solution_set = top_n_solutions(solution_set)
expanded_solutions = expand_solutions(solution_set)
```

SV IP model:

For a gene of interest, there are known structural variations. In the StARray caller, these are encoded as a set (“sv_configs”) of binary vectors [sv_vectors], in which 1 indicates the presence of an exon or intron and 0 indicates the absence of such. Other regions may be represented in the vector as well, including upstream and downstream of the gene of interest. An example sv_configs set showing three SV vectors is as follows:

$$[ [ \begin{matrix}1 & 1 & 1 & 1 & 1 & 1 & 1 & 1 & 1 & 1 & 1 & 1 & 1\end{matrix} ] \\ [ \begin{matrix}1 & 1 & 1 & 1 & 1 & 1 & 1 & 1 & 0 & 0 & 0 & 0 & 0\end{matrix} ] \\ [ \begin{matrix}0 & 0 & 0 & 0 & 1 & 1 & 1 & 1 & 1 & 1 & 1 & 1 & 1\end{matrix} ] ]$$

For CYP2D6, the above set of three sv_vectors might represent (i) a complete allele, (ii) a 3′ deletion, and (iii) a 5′ hybridization with CYP2D6, if reading through the binary vectors from top to bottom.

Each of the sv_vectors is also associated with a class, such as ‘complete allele’, CYP2D6*13, CYP2D6*68, etc. Each type of class may have one or more sv_vectors. For example, the sv_vectors [0, 0, 0, 1, 1, 1,] and [0, 0, 1, 1, 1, 1] both belong to the CYP2D6*13 class, indicating a CYP2D7-CYP2D6 hybrid. A vector cg_config is used to track the counts of these various sv_vector classes that are selected/called by the SV IP. If two complete alleles, one CYP2D6*13, and two CYP2D6*68 alleles are selected, for instance, then an example cg_config looks like [2, 1, 0, 2, 0], where the elements of cg_config correspond to the counts of (i) Complete, (ii) CYP2D6*13, (iii) CYP2D6*36, (iv) CYP2D6*68, and (v) CYP2D6*5, respectively.

The StARray SV caller also constructs a CNV vector, termed herein cnv_vector, from the input cnv.vcf file. An example of a CNV vector is [2, 0, 2, 0, 0, 3, 3], where each number signifies the copy number for an exon or intron (and/or other region(s) upstream or downstream of the gene of interest) derived from the cnv.vcf file. If an exon/intron is not represented in the cnv.vcf file, the value in its corresponding position of the CNV vector is 0 and its weight in the cost function becomes zero, as it does not contribute to the solution. Weighing is accomplished by a weight vector, termed herein v_{weight}. An example such v_{weight}=[p_{qual}, 0, p_{qual}, 0, 0, p_{qual}, p_{qual}], where a region's weight is either (i) the probability transformed Phred score from the cnv.vcf file, if the region is in the cnv.vcf file or (ii) 0, if it is not represented in the cnv.vcf file.

A vector, x_selection, with selection variables is also introduced. The x_selection vector is of the form [x₀, x₁, . . . , xₙ], where each xᵢ represents the copy number of a sv_vector from sv_configs in the solution.

With the above, an integer programming cost function is constructed as follows:

$$cost = \sum v ϵ \vert {v}_{weight} \times ( {cnv}_{vector} - ( {x}_{selection} \times s {v}_{configs} ) ) \vert$$

Substituting based on the above and using example values for v_{weight}, cnv_{vector}, x_{selection}, and sv_{configs}, the cost is expressed as:

$$\sum v ∈ \vert [ \text{.342} , 0 , \text{.89} , 0 , 0 , \text{.33} , \text{.9} ] \times ( [ 2 , 0 , 2 , 0 , 0 , 3 , 3 ] - [ \begin{matrix}{x}_{0} \\ {x}_{1} \\ {x}_{2}\end{matrix} ] [ \begin{matrix}1 & 1 & 1 & 1 & 1 & 1 & 1 \\ 1 & 1 & 1 & 1 & 1 & 0 & 0 \\ 0 & 0 & 0 & 0 & 1 & 1 & 1\end{matrix} ] ) \vert$$

in which the v_{weight} term is given by [0.342, 0, 0.89, 0, 0, 0.33, 0.9], the cnv_{vector} term is the cnv_vector and given by [2, 0, 2, 0, 0, 3, 3], the x_{selection} term is the x_selection vector and given by [x₀ x₁ x₂], and the sv_{configs} term is the sv_configs set and given by the three vectors [1 1 1 1 1 1 1], [1 1 11 10 0], and [0 0 0 0 1 1 1].

An additional component for consideration of parsimony and SV population frequency may be added to the cost function above in order to ensure that solutions with greater parsimony and more common SVs are returned first. As an example, this may be desired to ensure that two complete alleles will be preferentially returned rather than three hybrid alleles. An example representation of the cost function then becomes:

$$cost = \sum v ϵ \vert {v}_{weight} \times ( c n {v}_{vector} - ( {x}_{s e l e c t i o n} \times {sv}_{configs} ) ) \vert + PARSIMONY\_PENALTY \times \\ {sv\_frequency}_{vector} \times cg\_config$$

where the sv_frequency_{vector} term corresponds to a vector of the negative log of the population frequencies for the SVs.

Additionally, a constraint may be added to the SV IP model to ensure that a minimum number (e.g., 2) of SV vectors are selected for input to the Allele IP. An example such constraint to ensure that at least two SV vectors are selected is as follows:

$${\sum }_{{x}_{i} ∈ x\_select} {x}_{i} \geq 2$$

The SV IP returns the following:
x_{selection};
(x_{selection}×sv_{configs})=[cn_exon1, cn_intron1, cn_exon2, . . . , cn_exonₙ], which is the updated copy numbers of the intron and exon regions, termed cnv_vector_{updated};
Status: If the model was feasible;
cg_config—a vector indicating counts of SV classes that were selected; and
Solution Costs: A respective calculated likelihood of each of the SV solutions/candidates.

### Multi-Solution Selection:

This star-allele calling approach is unique at least in that it can return multiple alternate solutions for both the SV IP (discussed above) and Allele IP (discussed below) components as it explores the SV and Allele model spaces. This is achieved using a heuristic approach detailed herein.

To prevent redundancy of solutions generated by the SV IP model, the SV IP caller tracks the set of previously called solutions by maintaining a list, termed previous_solutions herein, of previously called cnv_vector_{updated} vectors.

The SV IP caller returns any given cnv_vector_{updated} once. For example, if a returned cnv_vector_{updated} vector has a value of [2, 2, 2, 3, 3], then this vector will not be returned again by the SV IP algorithm. The algorithm achieves this through a heuristic approach given as follows:

$$for each {cnv\_vector}_{updated} in previous\_solutions :$$

$$r = random\_float \_vector$$

$$current\_solution \_sum = sum ( r \times ( {x}_{selection} \times {sv}_{configs} ) )$$

$$previous\_solution \_sum = sum ( r \times {cnv\_vector}_{updated} )$$

$$add\_constraint ( difference ( current\_solution \_sum , previous\_solution \_sum ) > 0 )$$

The above approach adds the constraint that the sum of current cnv_vector_{updated} vector=(x_{selection}×sv_{configs}) does not equal the sum of any of the previously generated cnv_vector_{updated} vectors. The multiplication of the cnv_vector_{updated} by the random scaling vector r ensures that there will be a sum difference between solutions that are rearrangements of each other, i.e., (using the example above) [2, 2, 3, 3, 3] [3, 3, 3, 2, 2,].

The constraint add_constraint(difference(current solution_sum, previous_solution_sum)>0) is made linear as follows (as one example):

$$model += solution\_dif <= - \text{.00001} + 7 * b$$

$$model += solution\_dif >= \text{.00001} - 7 * ( 1 - b )$$

where b is a binary variable. For each previous solution, two new constraints and one variable are added to the model. The existence of the parsimony penalty ensures that the most parsimonious set of sv_vectors are returned with a cnv_vector_{updated} vector, removing the need to return multiple solutions with the same cnv_vector_{updated} vector.

### Solution Expansion:

Once all solutions have been generated, the individually called SV vectors are, in some embodiments, not utilized further by the algorithm. Instead, solution likelihoods are calculated at the higher level, SV classes selections, given by the cg_config vector. For CYP2D6, a cg_config vector might be =[2, 0, 1, 0, 0], indicating 2 complete alleles and 1 CYP2D6*68 allele.

After likelihood calculation (discussed below), the cnv_vector_{updated} and cg_config pairs are passed to a function that expands each selected config class to all SV vector combinations that are consistent with the solution cnv_vector_{updated}. All generated cnv_vectors and sv_vectors from the expanded solution set are passed to an Allele IP module/process.

### SV Solution Likelihood Calculation:

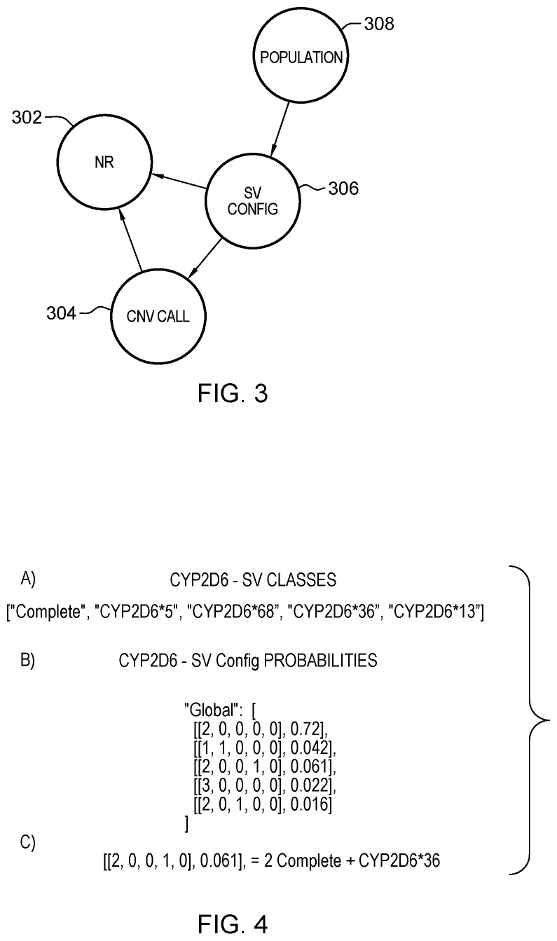

As the SV model space is explored, the log-likelihood of each SV solution is calculated. The sub-network (of FIG. 1) associated with the SV model space is shown in FIG. 3. Referring to the SV Config aspect 206, 306 in the sub-network, StARray calculates P(SV|Population), i.e., the probability of cg_config given a population specified by the user (as Population node 208, 308 in the sub-network). Recall that cg_config is a vector indicating the counts of SV classes. The likelihoods of known configurations of SV classes may be precomputed and statically stored in the StARray database. SV class configurations that are novel or low frequency may be calculated as the sum of the log probabilities of the individual SVs. An example negative log likelihood formula is given as: −log(P(CNV Call|SV))+−log(P(NR|SV))+−log(P(SV|Population)).

FIG. 4 shows an example of SV config probabilities for CYP2D6. Specifically, portion A) shows the list of 5 possible SV classes for CYP2D6, B) shows the probabilities of various (e.g., 5 different) SV class configurations for CYP2D6, and C) shows (i) the third such SV configuration, with 2 complete alleles and a *36 hybrid allele, and its associated probability, i.e., 0.061, for the global population.

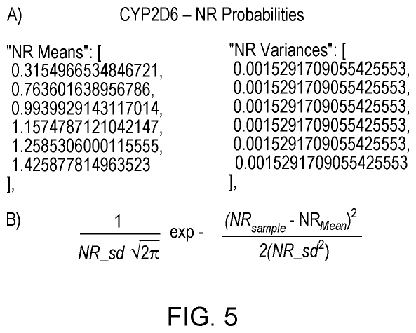

Referring to the NR node 310 in the sub-network of FIG. 3, NR values are provided by the CNV caller tool (that generated the input cnv.vcf data) for each region of the gene for which a copy number was calculated. Given an SV solution with its cnv_vector_{updated}, StARray calculates P(NR|SV), i.e., the probability of each NR value provided by the CNV caller tool. A Gaussian distribution (as one example) may be used to model the NR values. The means and variances for the NR values given the copy number value for each gene region may be obtained from the CNV caller tool's internal model. FIG. 5 shows example NR probability calculations, where A) shows the NR means and variances for copy numbers 0-5+ for CYP2D6, and B) shows the formula NR probabilities.

Finally, StARray determines P(CNV Call|SV), i.e., the probability of the CNV calls 204, 304 for the CNV caller tool given the selected SV config solution with its cnv_vector_{updated}. For each region of a gene for which there is a CNV call, StARray compares the call from the CNV caller tool to the updated value in cnv_vector_{updated}. Given the cnv_vector and the updated vector cnv_vector_{updated}, the probability of each gene region with a CNV call is calculated as follows:

$${cnv}_{vector [ i ]} = {cnv}_{{vector}_{updated} [ i ]} ; p = {phred}_{{probability}_{i}}$$

$${cnv}_{vector [ i ]} \neq {cnv}_{{vector}_{updated} [ i ]} ; p = 1 - {phred}_{{probability}_{i}}$$

### Star-Allele Calling (Star Allele IP):

The Allele IP for allele calling takes as input the expanded SV solutions/candidates consisting of cnv_vector_{updated} and sv_vector set pairs, and cg_config class counts. The Star allele calling is a multi-solution approach that uses integer programming to explore the allele Bayesian model space and find one or more sets of star-alleles that are feasible solutions/candidates for the given input vcf data. An example high-level pseudocode for the star allele IP is provided as follows:

```
hgvs_to_ vcf_map_masked = create_masked_map(input_vcf)
solution_setups = [ ]
for cnv_vector_{updated}, sv_vectors in sv_solutions:
  ab_allele_cn, ab_allele_quality =
    get_variant_cn_and_quality(cnv_vector_{updated}, input_vcf)
  allele_list, allele_vectors, cg_config =
    create_feasible_allele_vectors(ab_allele_cn)
  rare_allele_penalties = create_rare_allele_penalty_vector
    (allele_list, allele_vectors)
  solution_setup = tuple (ab_allele_cn, ab_allele_quality, allele_list,
    allele_vectors, cnv_vector_{updated}, cg_config, rare_allele_penalties)
  solution_setups.append(solution_setup)
  min_heap = new_min_heap
  for solution_setup in solution_setups:
    star_allele_solution = get_allele_solution(solution_setup)
    cost = get_solution_likelihood_from_Bayesian_graphical_model
      (star_allele_solution)
    min_heap.push (cost, solution_setup) # maintains sv_solutions,
      star-allele combinations in the order of the min cost
  solution_set = [ ]
  previous_solutions = [ ]
  while min_heap is not empty:
    solution_setup = head(min_heap)
    pop(min_heap)
    star_allele_solution = get_allele_solution(solution_setup)
    cost = get_solution_likelihood_from_Bayesian_graphical_model
      (star_allele_solution)
    if star_allele_solution.is_feasible and number_of_solutions < 10:
      solution_set.add(star_allele_solution)
      min_heap.push(cost, setup)
    previous_solutions.add(star_allele_solution)
```

The star-allele calling IP algorithm is a multi-solution approach and is accomplished using a min_heap. For each sv_solution (SV candidate) generated by the SV IP, the min_heap tracks the cost of the next star-allele solution/candidate that will be produced by processing a given sv_solution. The min_heap maintains at the top of the heap the sv_solution that will produce the most optimal star-allele solution/candidate. At each iteration, the multi-solution algorithm pops the sv_solution with the current lowest cost star-allele solution/candidate off the top of the heap. The multi-solution algorithm then finds a new star-allele solution/candidate with the current sv_solution. If this new solution is feasible, then it is added to the solution set and the sv_solution is added back onto the heap with the updated cost.

Various sub-functions of the multi-solution star-allele calling algorithm are detailed further as follows:

create_masked_map( ): The multi-solution allele calling algorithm begins by creating a masked mapping of probe identifiers to Human Genome Variation Society (hgvs) tags used by the known Pharmacogene Variation Consortium (PharmVar) and the Pharmacogenomics Knowledgebase (PharmGKB). The data from the mapping file, produced by a standalone variant-to-probe-identifier mapping utility for instance, is processed by the create_masked_map function to create a dictionary mapping probes to HGVS tags. Any variants that are not present in the array are masked from the dictionary to create a masked map.

get_solution_setups: This function encapsulates the following functions of the above pseudocode: ab_allele_cn, ab_allele_quality=get_variant_cn_and_quality (cnv_vector_{updated}, input_vcf); allele_list, allele_vectors, cg_config=create_feasible_allele_vectors(ab_allele_cn); rare_allele_penalties create_rare_allele_penalty_vector (allele_list, allele_vectors). The function obtains a solution setup for each of the sv_solutions produced by the SV IP algorithm. Data produced in this aspect includes a list (allele_list) of feasible star alleles, boolean star allele vectors (allele_vectors) representing the presence or absence of variants/reference alleles in that star-allele, a vector (ab_allele_cn) with the estimated copy number of the variants/reference alleles in the sample, the quality values (ab_allele_quality) of variants/reference alleles in the sample, a cnv_vector_{updated} for the sv solution, cg_config (the structural class counts for the sv_solution), and a vector (rare_allele_penalties) indicating which star-alleles in the allele_list are rare alleles along with associated penalties.

Within the get_solution_setup function, several sub-functions exist to obtain the described data for the Allele IP solution. The sub-functions are described as follows:

get_variant_cn_and_quality( ): This function generates the ab_allele_cn and ab_allele_quality vectors which are the estimated variant/reference allele copy numbers and variant/reference allele quality values, respectively. An example pseudocode for generating variant copy number and quality vectors ab_allele_cn and ab_allele_quality using the B-allele Frequency (BAF) is as follows:

```
ab_allele_cn = 2 * length(hgvs_to_vcf_map_masked) # accounting for
  both the variants and the reference allele value
ab_allele_quality = 2 * length(hgvs_to_vcf_map_masked)
for each, i, variant in enumerate(hgvs_to_vcf_masked):
    vcf_record = get_vcf_record(variant)
    variant_baf = vcf_record[‘BAF’]
    variant_qual = vcf_record[‘GS’] # this can also be ‘GQ’ depending
      on the snv.vcf type
    # variant
    ab_allele_cn[i] =
      cnv_vector_{updated}[get_intron/exon_of_variant(variant)] *
      variant_baf
    # reference allele
    ab_allele_cn[i + length(hgvs_to_vcf_masked)] =
      cnv_vector_{updated}[get_intron/exon_of_variant(variant)] * (1 −
      variant_baf)
    ab_allele_quality[i] = logit(variant_qual)
    ab_allele_quality[I + length(hgvs_to_vcf_map_masked)] =
      logit(variant_qual)
Return ab_allele_cn, ab_allele_quality
```

The ab_allele_quality are the logit transformed GenCall scores associated with each variant/reference allele obtained from the input snv.vcf.

create_feasible_allele_vectors( ): This function takes the sv_vectors from a sv_solution and generates feasible star-allele vectors from them. Recall that a sv_vector indicates structural variation configurations such as ‘complete’ or CYP2D6*36, as examples. For each sv_vector, all feasible underlying alleles are considered. For example, CYP2D6*68 might have CYP2D6*4 and CYP2D6*10 as feasible under-lying alleles. The create_feasible_allele_vectors function generates the star-allele vectors for star-alleles that are feasible given the variant/reference allele copy number coverage and quality. The calculation of the feasibility of a star allele given the sample data is described below.

If a variant belonging to a star-allele is not present within the sample, then the star-allele is not a feasible component of the star-allele solution. However, a low-quality variant may be erroneous within the vcf sample. To address this, the variant qualities (quality values/scores from the input snv.vcf) may also be taken into consideration. As examples, for a star-allele to be considered feasible, the quality scores of the variants belonging to the star-allele but that are not present in the sample are to be less than a user-provided threshold (quality_cutoff) and at least one variant belonging to the star-allele is to be present. To be considered present in the sample, a variant is to have a copy number value in ab_allele_cn greater than another user-provided threshold (coverage_cutoff). Reflection of a variant being present in the sample is determined by the ab_allele_cn vector, which holds the copy numbers that StARray determined for the variants; by way of specific example and not limitation, if a variant, i, has ab_allele_cn[i]=0.3 and this is less than the coverage_cutoff (of, say, 0.9), then it is considered to not be present in the sample. And if the quality value of variant i is relatively low, for instance 0.7, then variant i is disregarded as a requisite variant to have observed in the sample for purposes of considering the star-allele to be a possible solution. If instead the variant i has a relatively high-quality value, of 0.999 for example, then the candidate star-allele may be considered to be infeasible as a variant since a high-quality value variant belonging to that star-allele is not present. Meanwhile, in examples, StaARray also needs at least one variant belonging to a candidate star-allele to present in the sample for the star-allele to be considered, and quality is not considered in this aspect. If a different variant j associated with the candidate star-allele has ab_allele_cn[i]=1.3>coverage_cutoff=0.9 then it is considered to be present in the sample and meets this criteria. The foregoing therefore provides two separate tests in this regard.

Example pseudocode for determining a star-allele's feasibility is shown as follows:

```
conflicting_variant = False
for variant in star-allele:
  if ab_allele_cn[variant] <
    coverage_cutoff and ab_allele_quality[variant] > quality_cutoff:
    conflicting_variant = True
    break
at_least_one_variant_in_sample = False
for variant in star-allele:
  if ab_allele_cn[variant] >= coverage_cutoff:
    at_least_one_variant_in_sample = True
```

if not conflicting_variant and at_least_one_variant_in_sample:
   return true #the allele is feasible/else return False

Once a star-allele is determined to be feasible, an allele_vector is generated for that allele. For instance:

$$allele\_vector = [ \begin{matrix}0 & 0 & 0 & 1 & 0 & 0 & 1 & 1 & 1 & 0 & 1 & 1 ]\end{matrix}$$

where:
length(allele_vector)=2*length(hgvs_to_vcf_map_masked); and
allele_vector[i]=1, if variants[i]∈star_allele, else allele_vector[i]=0, for i in range (0, length(hgvs_to_vcf_map_masked)); and
allele_vector[i+length(hgvs_to_vcf_map_masked)]=1, if
reference_allele(variants [i])∈star_allele, else allele_vector[i]=0, for i in range(0, length(hgvs_to_vcf_map_masked)).

The allele vector takes into consideration both the variants and reference alleles present in the star-allele. Each underlying star allele is constructed regarding the overlying sv_vector. If the sv_vector is complete, then the entire star-allele will be represented in the allele_vector. If the sv_vector is a hybrid or partial allele, then variants/reference alleles falling within the missing portion of the sv_vector will be set to 0 in the allele_vector.

Star-alleles may have optional core variants associated with them. For example, this is true of CYP2D6*4, which is defined by one core variant but can also have additional core variants associated with it. Note that these are not minor star variants but are Core Variants. These optional variants may be indicated by a 2 in the allele_vector, which will allow them to be distinguished in the allele variable creation in the allele IP programming.

A final allele_vector might therefore look like:

$$allele\_vector = [ \begin{matrix}0 & 0 & 2 & 1 & 0 & 0 & 1 & 1 & 0 & 0 & 1 & 1 ]\end{matrix}$$

create_rare_allele_penalty_vector( ): This function creates the rare_allele_penalties vector. For each of the feasible star-alleles, a respective rare_allele_penalty is set to (1−population_frequency(star-allele)), where population_frequency is the population_frequency of the star-allele given a user-selected population.

### Star-Allele Calling IP Model:

Now that all the sv_solutions have produced a solution setup for the Star-allele calling, a star-allele calling model/function is performed. The star-allele calling function takes as input the generated solution_setups consisting of allele_list, allele_vectors, ab_allele_cn, ab_allele_quality, cnv_vector_{updated}, cg_config, rare_allele_penalties.

The main form of the cost function of the star-allele IP model to call star alleles is as follows:

$$\sum \frac{{v}_{i}}{cnv\_vector \_updated [ region [ {v}_{i} ] ]} ∈ \vert ab\_allele \_cn - ( allele\_selection \times allele\_vectors ) + allele\_aux \_variants ( variant\_v ) \vert + RARE\_ALLELE \_PENALTY X ( rare\_allele \_vector \times allele\_selection )$$

The allele_selection vector is a vector indicating how many copies of a star-allele are in the star-allele solution. The allele_aux_variants are variables that represent the presence or absence of the optional major-star variants (i.e., Core Variants) for each allele_vector.

An example of ab_allele_cn is [1.82, 2.01, 2.33, 1.92, . . . , 1.89, 3.2, 2.9, 3.01]. An example of allele_selection is [x₁, x₂, x₃, . . . , xₙ]. An example of allele_vectors is:

$$[ [ \begin{matrix}1 & 1 & 1 & 1 & 1 & 1 & 1 & 1 & 1 & 1 & 1 & 1 & 1\end{matrix} ] \\ [ \begin{matrix}1 & 1 & 1 & 1 & 1 & 1 & 1 & 1 & 0 & 0 & 0 & 0 & 0\end{matrix} ] \\ [ \begin{matrix}0 & 0 & 0 & 0 & 1 & 1 & 1 & 1 & 1 & 1 & 1 & 1 & 1\end{matrix} ] .$$

An example of allele_aux_variants (variant_v) is

$$[ 0 , {v}_{1 \_ alt} , {v}_{10 \_ alt} , 0 , 0 , … , {v}_{{1}_{ref}} {v}_{10 \_ref} ] .$$

The star-allele calling model creates the necessary allele_selection and allele_aux_variants variables needed for the star-allele calling. The star-allele IP variable creation is described as follows:

create_allele_variables( ): This function takes the allele_vectors, ab_allele_cn (the variant and reference allele copy numbers), and the structural variant class counts, i.e., cg_config, as input. For each star-allele belonging to an overlying config class in cg_config, such as a complete allele, a variable xᵢ is created and added to allele_selection.

If an optional core variant is indicated in the star-allele allele_vector (e.g., indicated by 2), then additional variables are introduced. For each optional core variant in a star-allele, two variables, vₐₗₜ and v_{ref}, are introduced. These variables track the number of copies of the optional alternative allele and the reference allele, respectively, and are maintained in the vector allele_aux_variants, with one entry per star-allele. The allele_aux_variants vector can be reformatted into an additional vector (variant v), which tracks the optional major star-variants by variant, rather than by star-allele, for use in the cost function.

Three constraints may be added to the star-allele IP model as follows:
1. For each vₐₗₜ and v_{ref} pair belonging to star-alleleᵢ, vₐₗₜ+v_{ref.}=xᵢ in allele_selection
2. For all star-alleles=star-allele1, star-allele2, star-allele3, . . . , star-alleleN belonging to a cg_configᵢ class, x₁+x₂+x₃+ . . . +xₙ=cg_config[i]
3. For all ab_allele_cn[i], if ab_allele_cn[i]>=coverage_cutoff and ab_allele_quality[i]>=quality_cutoff: count(alleles_in_solution_containing_variant_i)>=1 #If a variant is present in the sample with a quality value greater than the cutoff, then it must be covered by one of the star-alleles in the generated solution

The above three constraints ensure, respectively, that (i) for each optional major-star variant in a given star-allele, the sum of the ref and alt allele copy numbers equals the number of copies of the star-allele, (ii) the number of star-alleles selected in each cg_config class equals the count of the cg_config class called by the SV IP, and (iii) the quality of variant/reference alleles that appear in the sample but not in the solution is less than a user-provided threshold.

### Star-Allele Call Likelihood Calculation:

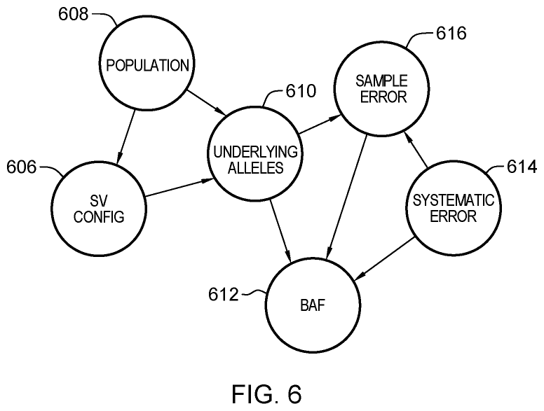

As the allele model space is explored, the log-likelihood (i.e., as negative log-likelihood) of each SV candidate is calculated. The sub-network associated with the Allele model space is shown FIG. 6. As discussed above, the probability formula is given as P(Underlying Alleles|SV Config, Population)P(BAF|Underlying Alleles, Sample Error, Systematic Error)P(Sample Error|Underlying Alleles, Systematic Error)P(Systematic Error). The negative log likelihood formula is therefore −log(P(Underlying Alleles|SV Config, Population))+−log(P(BAF|Underlying Alleles, Sample Error, Systematic Error))+−log(P(Sample Error|Underlying Alleles, Systematic Error))+−log(P(Systematic Error)).

Referring to FIG. 6, population 608 is the population (see also 208, 308) used for calculating the frequencies. The SV config node 606 represents the probability of one of the solutions generated by the SV integer programming. The underlying alleles node 610 represents the probability of an underlying star-allele diplotype, given the SV config solution provided by the SV integer programming. This probability of the underlying star-allele diplotype, P(Underlying Alleles|SV Config, Population), is calculated by dividing (i) the probability of a pair of given star-alleles, hybrid alleles, or tandem alleles by (ii) the probability of all configurations containing the allele of interest.

The BAF node 612 in FIG. 6, like the NR value discussed above, is modeled as a Gaussian distribution:

$$\frac{1}{BAF\_sd \sqrt{2 π}} exp - \frac{{( B A {F}_{sample} - B A {F}_{Mean} )}^{2}}{2 ( {BAF\_sd}^{2} )}$$

in which the BAF mean and standard deviation are obtained from a clustering algorithm (e.g., GenTrain clustering) based on a set of training samples. The BAF probability, P(BAF|Underlying Alleles, Sample Error, Systematic Error), may be calculated based on the presence of Sample and Systematic error in one of the following approaches:

### Approach 1:

$$1 ) If {Sample}_{Error} = False , {Systematic}_{Error} = False : \\ {BAF}_{sd} = {BAF}_{sd} \\ P ( {Sample}_{Error} ) = {gs}_{probability} \\ P ( {Systematic}_{Error} ) = P ( sample\_variant \_frequency )$$

$$2 ) If {Sample}_{Error} = True , {Systematic}_{Error} = False : \\ {BAF}_{sd} = \text{.1} \\ P ( {Sample}_{Error} ) = 1 - {gs}_{probability} \\ P ( {Systematic}_{Error} ) = P ( sample\_variant \_frequency )$$

$$3 ) If {Sample}_{Error} = True , {Systematic}_{Error} = True : \\ P ( BAF ) = 1 \\ P ( {Sample}_{Error} ) = 1 - {gs}_{probability} \\ P ( {Systematic}_{Error} ) = 1 - P ( sample\_variant \_frequency )$$

The minimum total cost of these three states is selected as the state configuration and cost.

### Approach 2:

$$1 ) If {Sample}_{Error} = False , {Systematic}_{Error} = False : \\ {BAF}_{sd} = {BAF}_{sd} \\ P ( {Sample}_{Error} ) = {gs}_{probability} \\ P ( {Systematic}_{Error} ) = 1$$

$$2 ) If {Sample}_{Error} = True , {Systematic}_{Error} = False : \\ {BAF}_{sd} = \text{.1} \\ P ( {Sample}_{Error} ) = 1 - {gs}_{probability} \\ P ( {Systematic}_{Error} ) = 1$$

$$3 ) If {Sample}_{Error} = True , {Systematic}_{Error} = True : \\ P ( {Sample}_{Error} ) = 1 - {gs}_{probability} \\ P ( {Systematic}_{Error} ) = 1 \\ P ( variant present in solution \vert {Systematic}_{Error} = True ) = variant\_population \_frequency \\ P ( variant not present in solution \vert {Systematic}_{Error} = True ) = 1 - variant\_population \_frequency$$

Sample error is determined to be present if P(BAF|No sample error)<0.01 or Systematic error is present. Systematic error is determined to be present if P(sample_variant frequency)<user_provided_threshold. If systematic error is detected, then the probability of the presence or absence of the variant of interest in the solution is determined instead of P(BAF) according to population frequency.

The Sample error node 616 of FIG. 6 represents the probability of the sample error, and is either 1−gs_probability or gs_probability depending on the absence or presence of sample error. The gs_probability is the logit transformation 1/(1+exp(−(GenCall score−0.14)*140)) of a GenCall score from the snv vcf file. It is noted that 0.14 and 140 are just example values; these parameters may be adjusted as desired for optimization purposes.

Occasionally, one or more variants in a batch of samples may be impacted by genotyping batch effect or clustering error. To detect this type of error, the frequencies of sample variants are compared to known reference population frequencies. The Systematic error node 614 of FIG. 6 represents the probability of systematic error. Variant sample frequency can be compared to the population frequency to detect failed clusters.

### Bayesian Graphical Model Likelihood:

The final (‘overall’) likelihood for each star-allele call determined from the Bayesian graphical model is a composite (such as the sum) of the respective SV sub-network log likelihood and the respective allele sub-network log likelihood for that call.

It is seen that potentially multiple alternative calls might result from the above, each with a respective overall likelihood. The multiple possible calls can be output along with their likelihoods. Such output could optionally be provided as a ranking of those calls based on their likelihoods, and the ranking of the possible calls could be used in any manner desired, for instance for filtering purposes.

Table 1 below depicts example results of aspects described herein as applied to the CYP2D6 PGx gene and in comparison to results of the DRAGEN® NGS Star Allele Caller tool (DRAGEN is a registered trademark of Illumina, Inc.) to genotype CYP2D6 from a whole-genome sequencing (WGS) BAM file.

TABLE 1

| Caller | StARray Accuracy* | No Solution |
|---|---|---|
| StARray | 140; (100%) solutions | 1 (0.7%); |
| | compared = 140 | n = 141 |
| DRAGEN NGS | 121; (93.1%) solutions | 11 (7.8%); |
| Star Allele | compared = 130 | n = 141 |
| Caller | 3 match 1^{st} alternative | |
| | solution | |
| | 124; (95.4%) solutions compared = 130 | |
| Existing Star | 108; (83.7%) solutions | 12 (8.5%); |
| Allele Caller | compared = 129 | n = 141 |
| on BeadArray | | |

| Caller | TSI Accuracy* |
|---|---|
| DRAGEN NGS | 101; (84.1%) solutions compared = 120 |
| Star Allele Caller | 21 samples excluded due to no solutions |
| | for one or both algorithms |

*Accuracy was evaluated for samples where both algorithms that are being compared had a call. 141 samples from wave 1 evaluated for CYP2D6

Looking into the 9 samples that had star-allele solutions that were discordant between DRAGEN® NGS Star Allele Caller tool and StARray:
For 3 samples, the next alternate StARray solution was the same as the DRAGEN® NGS Star Allele Caller tool;
For 204619760003_R07C01 and 204619760012_R03C01, there is a variant exclusive to CYP2D6*117 that is being called with very high GS scores (0.7341 and 0.7339 respectively). Both the existing PGx caller in Illumina's Array PGx Analysis pipeline and StARray call *117 but the DRAGEN® NGS Star Allele Caller tool does not;
For 204619760008_R03C01, the variant associated with CYP2D6*22 is not genotyped in the array due to very low quality (e.g., GS score ˜0.03). The PGx caller in Illumina's Array PGx Analysis pipeline and StARray call this as *1 instead of CYP2D6*22 as the DRAGEN® NGS Star Allele Caller tool does;
For 204619760012_R08C01, there is more of a discrepancy on the CNV call. The CNV region that led to a discordant call was CNV:CYP2D6.p5:chr22:42130886:42131379. This had a relatively high-quality value of 28, leading both the existing PGx caller in Illumina's Array PGx Analysis pipeline and StARray to have a slightly different copy number than the DRAGEN® NGS Star Allele Caller tool. (*4+*68/*4 vs *4+*68/*4+*68);
For 204619760010_R07C01, the BAF value supported the call of two *90 alleles by StARray;
For 204619760005_R02C01, there is more of a discrepancy on the CNV call. The CNV region that led to a discordant call was CNV:CYP2D6.exon.9:chr22:42126498:42126752. This had a moderate quality value of 19, leading to StARray to have a slightly different copy number than the DRAGEN® NGS Star Allele Caller tool (*10,*36, *36, *36 vs *10+*36/*10+*36)

In some examples, log likelihood scores, for instance the score corresponding to each star-allele call determined from the Bayesian graphical model, can be converted into representations that are more convenient for interpretation, downstream usability, or other purposes. For example, such likelihoods could be converted to a posterior probability by normalizing each score based on the scores for the collection of candidate solution scores (e.g., the sum of the scores of all candidate solutions), to produce a posterior probability value between 0 and 1. This may be preferred to the raw log likelihood value, in some situations.

In some examples, a caller for single variants is implemented that identifies genotype variant calls from the input single nucleotide variant data (e.g., snv.vcf) and calculates an associated log likelihood probability using the Bayesian graphical model as described above, but without the integer programming aspects, to report the log likelihood of that variant call.

Results of processing described herein can be output in any desired format. As noted above, calls can be output, along with their likelihoods, and ranked based thereon. In examples, a set of candidate star allele solutions, and a ranking of those solutions, potentially fitting the array data can be output for each sample.

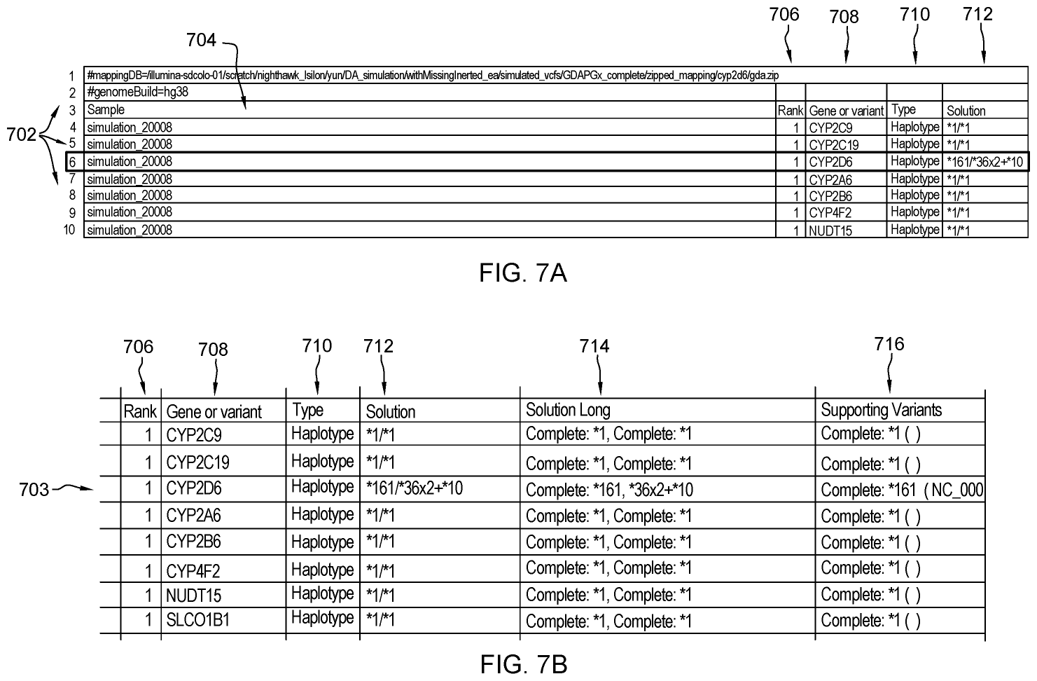

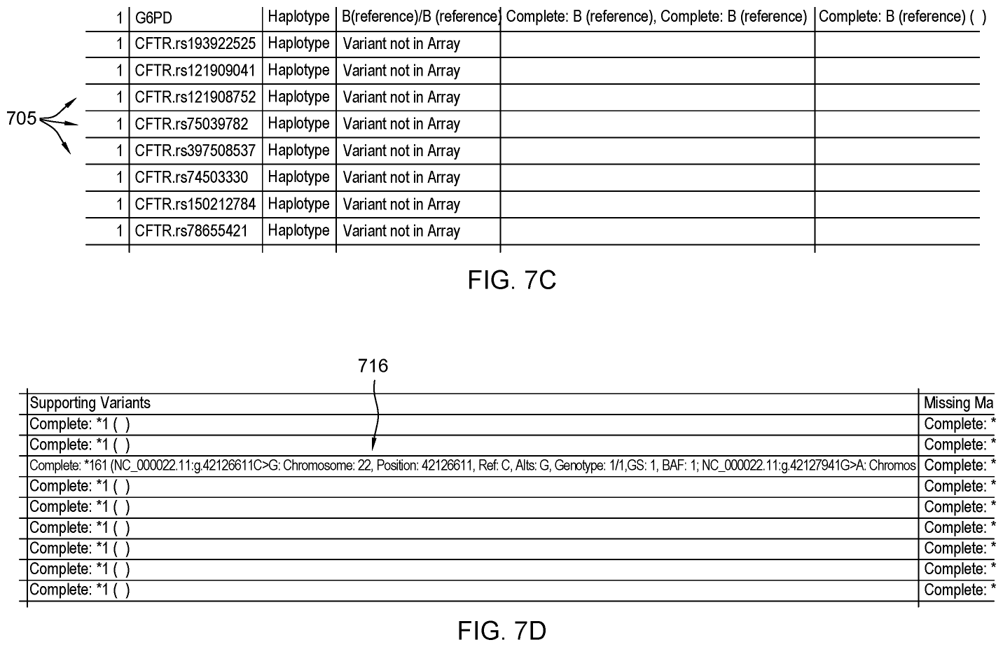

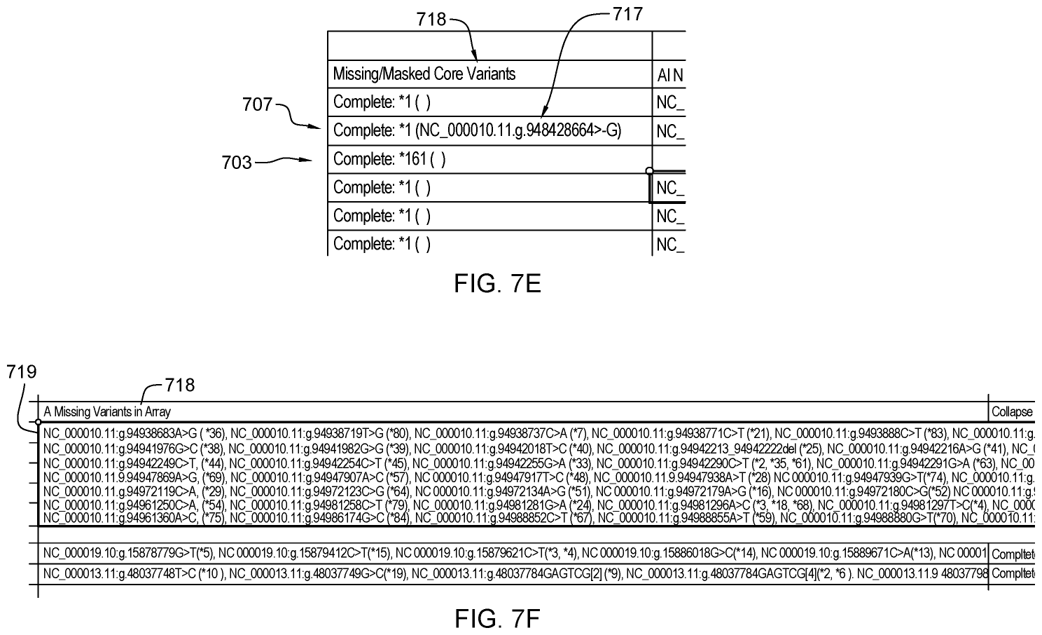

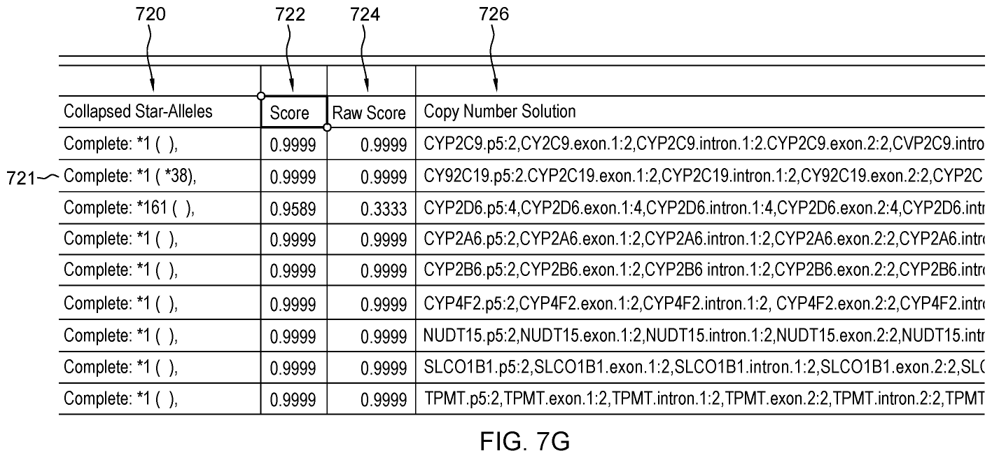

FIGS. 7A-7G depict example results output for candidate solutions, in accordance with aspects described herein. Output is provided, in these examples, in the form of a table/spreadsheet with most rows corresponding to samples and most columns corresponding to different result data for each of those samples. The output can be displayed on a graphical user interface for viewing and interaction by a user. Each of FIGS. 7A-7G depict portions of an example spreadsheet shown on a graphical user interface.

Referring initially to FIG. 7A, 10 rows 702 of the output are shown for illustration. The first two rows provide header information for the data, and the third row contains headings/names for each column. The fourth through tenth rows show partial results for 7 samples. In practice, that results output would typically include results for many more samples, for example hundreds or thousands of samples.

Different columns provide different types of results data. Shown in FIG. 7A are data indicating Sample name 704, determined sample Rank 706, indication of Gene or Variant 708, Type 710, and Solution 712. In FIG. 7A, row 6 is highlighted, corresponding to the CYP2D6 gene.

Further details are shown in FIG. 7B, which depicts additional columns for Solution Long 714 data and Supporting Variants 716 data. Referring to the row identified by 703 (corresponding to the CYP2D6 gene) in FIG. 7B, the Type 710 is appropriately indicated as Haplotype. In this regard, the caller distinguishes between haplotype calls (e.g., star alleles) and variant calls, or ‘simple variants’. FIG. 7C depicts example rows 705 of results data for variant samples, in which the variant name is provided and the sample is identified as a simple variant.

Referring back to FIG. 7B, and specifically to the results for the CYP2D6 gene sample, the Solution 712 provides a simple representation of the solution, which in this example indicates a complete allele (*161) and tandem allele (*36×2+*10). Solution Long 714 indicates a longer-form solution that conveys more detailed information about the structural variants, as applicable. For CYP2D6, this indicates “Complete: *161, *36×2+*10” to indicate a complete *161 allele and a tandem allele *36×2+*10.

The Supporting Variants data 716 provides any reported supporting variants. For any given star allele identified, there may or may not be all variants present in the array. The Supporting Variants 716 data reports the variants that were detected in the array. Referring to FIG. 7D, a portion of the Supporting Variants 716 for the CYP2D6 gene are shown along with some key information about that variant. The reported variants lead with those corresponding to the *161 allele. Information provided includes (using the first reported variant in this example) chromosome location (22), position (42126611), reference value (C), variant value (G), and other information. Additionally provided is the actual genotype (e.g., 1/1) that was found in the BCF file, the GS score (1), which is a score indicating the confidence of the variant call, and the BAF value (1), which is the B-allele frequency that can also be used for determining whether or not the call made was accurate. The supporting information for the star alleles provided by the caller can be very useful for debugging and other purposes.

Referring to FIG. 7E, additional results information that might be useful for debugging (or other purposes) is Missing/Masked Core Variants data 718, providing any missing or masked core variant from the array. Using the CYP2D6 gene example from row 703, no variants were missing (“Complete: *161( )”). Row 707, corresponding to results for the CYP2C19 gene, indicates that the Core Variant identified by 717 was missing from the array. The results for supporting variants and missing variants provides users an easy way to determine what was and what was not present in the array to support the corresponding star allele call.

Referring to FIG. 7F, a column for All Missing Variants in Array 718 reports, for the associated sample (from row 719 here) all PGx-related variants that are missing from the array (or filtered, for instance from quality control processing due to low quality, as an example) and the star allele that is potentially affected by that. The first variant reported corresponds to the *36 star allele, and the second corresponds to the *80 start allele, as examples.

As shown in FIG. 7G, additional columns for Collapsed Star-Alleles 720, Score 722, Raw Score 724, and Copy Number Solution 726 are provided. Regarding Collapsed Star-Alleles, it is sometimes difficult or impossible to distinguish one star allele from another when there are missing variants from the array. Therefore, the Collapsed Star-Alleles data for each sample reports all star alleles that are collapsed into the same haplotype, listed out beginning with the most-frequent star allele and followed by the least/less frequent star alleles enclosed in parentheses. Data for the sample of row 721 indicates *1 as the most frequent star allele but also indicates the less-frequent *38 star allele.

The Score data 722 and Raw Score data 714 present the Bayesian graphical model likelihood log likelihood transformed into the posterior probability and either (i) accounting for the population prior probability (presented as Score 722) or (ii) not accounting for the population prior probability (presented as Raw Score 724). In either column, a higher value indicates a higher probability in the likelihood of the called solution.

The Copy Number Solution data 726 presents a prediction of the copy number for each exon and intron within the indicated gene.

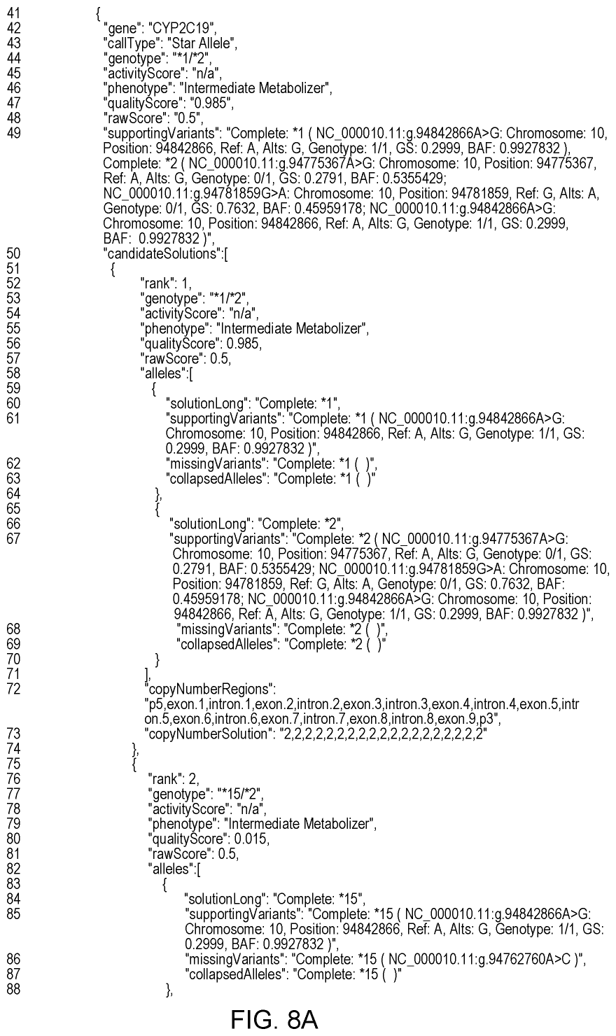

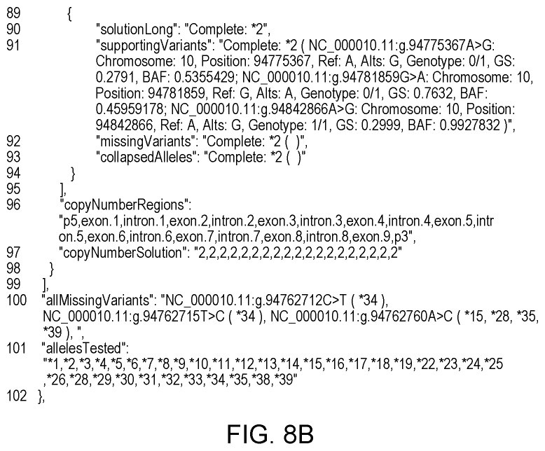

The results output of FIGS. 7A-7G is provided in the form of a table, but other formats are possible. FIGS. 8A-8B depict additional example results output for candidate solutions, in accordance with aspects described herein, and more specifically a portion of results output in the JSON (JavaScript Object Notation) format. As above, the data provided in the JSON example of FIG. 8A-8B is provided per-sample. A portion of the JSON output for one sample, the CYP2C19 gene, is shown in FIGS. 8A-8B.

In addition, the data for each star allele indicated is annotated to indicate a metabolizer status (“phenotype”). In the PGx space, two well-known public guidelines that include metabolizer status indications are those promulgated by the Clinical Pharmacogenetics Implementation Consortium (CPIC) and the Dutch Pharmacogenetics Working Group (DPWG). The CPIC guidelines are used in this example, which includes metabolizer statuses of Ultrarapid, Normal, Intermediate, and Poor, as examples.

The results output is presented in the form of a field/type together with the value for that field. Referring to FIGS. 8A-8B, the record shown indicates the fields with corresponding values, including “gene” indicating the CYP2C19 gene; “callType” indicating a star allele call; “genotype” indicating overall genotype of “*1/*2”; “activityScore” assigned to the allele, if present/applicable; and “phenotype” indicating a mapping to the “Intermediate Metabolizer” status. Additional fields corresponding to fields discussed above are provided, including those for score (“qualityScore”), raw score (“rawScore”), supporting variants (“SupportingVariants”), and candidate solutions (“candidateSolutions”). With respect to candidate solutions, data for two solutions is shown in this example to indicate two solutions with different star alleles—*1/*2 vs. *15/*2 in this example. The candidate solutions portion provides the top ranked solutions (e.g., two in this example but more could be provided). For each candidate solution, various information is provided, for instance some or all of the information discussed above such as genotype, activity score, phenotype, quality score, raw score, and allele information for the various alleles, including the Solution Long, Supporting Variants, Missing Variants, and Collapsed Alleles information.

The overall “phenotype” provided above the candidate solutions (“Intermediate Metabolizer” here) could be an aggregation of the phenotype(s) indicated for the individual candidate solutions; here there is a consensus (“Intermediate Metabolizer”) between the two different solutions corresponding to different genotypes but a common phenotype. The overall phenotype to call in situations with varying phenotype across the solutions could follow any desired approach. One such approach is to take the phenotype of the top-ranked solution, as an example.

Although not shown, additional information included in the JSON output for each gene could be listings of all missing variants, and a listing of alleles tested, as examples.

It is seen that the JSON output of FIG. 8A-8B can include the same information as was provided in the examples of FIGS. 7A-7G, plus other/additional information, for instance aggregated call information to include a mapped metabolizer status/phenotype and activity score, for example.

JSON and similar formats can employ syntax-based coloring to the different types of data included, for instance providing data types/fields in one color and values for those fields in another color. Any other highlighting, coloring, or other visual indications to distinguish some data from other data could be used.

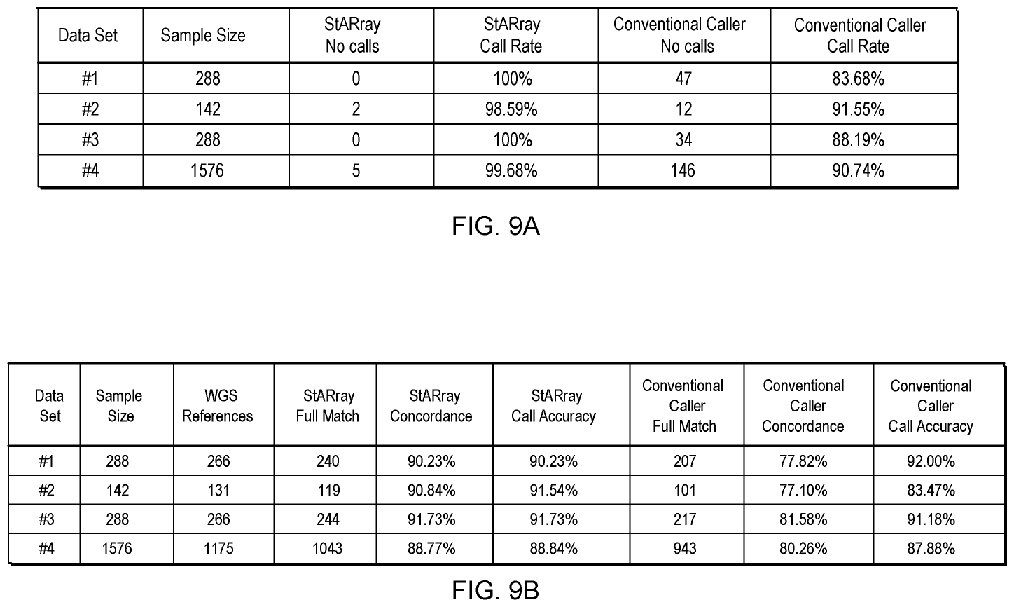

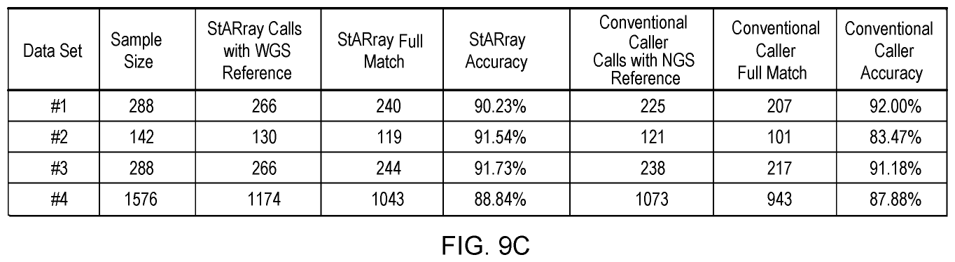

FIG. 9A-9C depict example comparison results from testing the StARray approach described herein against a conventional caller. The comparison results are presented in the form of tables in these Figures. The testing was conducted using four different testing datasets (#1, #2, #3, and #4). The datasets ranged in size from 142 samples (#2) to 1576 samples (#4), and utilized Coriell cell line samples. Each dataset was provided as input to the conventional caller and, separately, to a caller implementing the StARray approach described herein.

Referring to FIG. 9A, the table includes six columns. From left-to-right, the first column identifies the data set, the second column indicates the sample size of the data set, the third and fourth columns indicate the number of no calls and call rate using the StARray approach, and the fifth and sixth columns indicate the number of no calls and call rate using the StARray approach. The call rates reflect samples run on Global Diversity Array with Enhanced PGx and evaluated for the CYP2D6 gene.

As seen from the results in FIG. 9A, the StARray caller provides significant improvements over the conventional caller in terms of lower numbers of no-calls, and consequently higher call rates (and lower no-call rates).

Referring to FIG. 9B, these results indicate concordance and include array caller no-calls in the comparison. The table includes nine columns: from left-to-right, the first and second columns identify the data set and sample size of the data set as in FIG. 9A. The third column indicates the number of Whole Genome Sequencing (WGS) references (comparisons) from the sample set. The fourth, fifth, and sixth columns indicate the number of ‘full matches’, i.e., the number of WGS references that the caller fully matched, the concordance (full matches divided by the number of WGS references), and call accuracy, respectfully, for the StARray caller. The seventh, eight, and ninth columns indicate the number of ‘full matches’, i.e., the number of WGS references that the caller fully matched, the concordance (full matches divided by the number of WGS references), and the call accuracy, respectfully, for the conventional caller. The concordance reflects samples run on Global Diversity Array with Enhanced PGx and evaluated for the CYP2D6 gene. Not all samples had a whole-genome sequence (WGS) reference and/or had a no call in the WGS data. As seen from the results in FIG. 9B, the StARray caller provides significant improvements over the conventional caller in terms of the number of full matches and concordance.

Referring to FIG. 9C, these results indicate caller accuracy and do not include no calls in the comparison. The table includes eight columns: from left-to-right, the first and second columns identify the data set and sample size of the data set as in FIGS. 9A and 9B. The third, fourth, and fifth columns indicate, respectively: the number of called diplotypes with the WGS reference, the number of ‘full matches’ (same as the fourth column of FIG. 9B), and the accuracy (full matches divided by the number of called diplotypes), for the StARray caller. The sixth, seventh, and eighth columns indicate, respectively: the number of called diplotypes with the WGS reference, the number of ‘full matches’ (same as the sixth column of FIG. 9B), and the accuracy (full matches divided by the number of called diplotypes), for the conventional caller. The accuracy reflects samples run on Global Diversity Array with Enhanced PGx and evaluated for the CYP2D6 gene. Again, not all samples had a whole-genome sequence (WGS) reference and/or had a no call in the WGS data. As seen from the results in FIG. 9C, the StARray caller provides significant improvements over the conventional caller in terms of accuracy.

Partial matches and mismatches may also be tracked, and an overall accuracy (incorporating numbers for full matches, partial matches, and mismatches) could be determined, if desired.

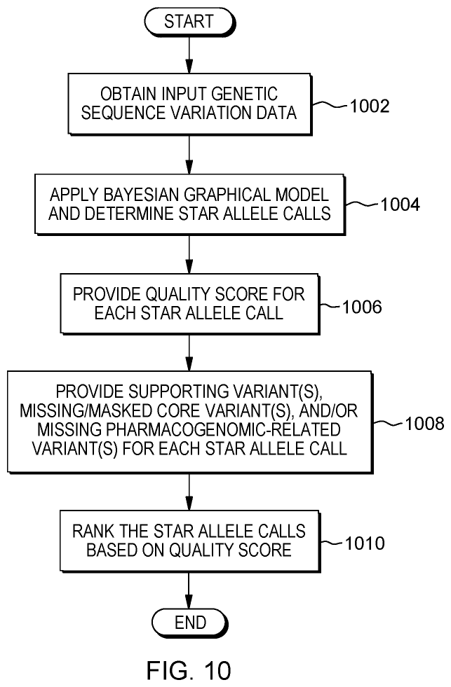

Accordingly, FIG. 10 depicts an example process for star allele calling in accordance with aspects described herein, for instance a process for determining pharmacogenomics gene star alleles using high-throughput targeted genotyping. The process may be executed, in one or more examples, by a processor or processing circuitry of one or more computers/computer systems, such as those described herein. For instance, code or instructions implementing the process of FIG. 10 may be part of modules of software/computer program(s).

Referring to FIG. 10, the process obtains (1002) input genetic sequence variation data from a high-throughput genotyping platform based on a pharmacogenomic genotyping of a sample. By way of example, the high-throughput genotyping platform includes a microarray-based genotyping platform. The input genetic sequence variation data can include genotype data and copy number variant call data, for instance. In some embodiments, the genotype and copy number data includes B-allele frequency (BAF) and log R ratio data.

The process also applies (1004) a Bayesian graphical model to determine a plurality of different star allele calls corresponding to the sample. For example, the applying the Bayesian graphical model uses multi-solution integer programming to explore a model space of the Bayesian graphical model in (i) a first phase that includes structural variant (SV) candidate identification and (ii) a second phase that includes star allele candidate identification based on the SV candidate identification, to determine the plurality of different star allele calls.

In embodiments, the first phase identifies a plurality of SV candidates and evaluates, for each SV candidate of the plurality of SV candidates, a cost of the SV candidate. The cost of the SV candidate could include a log transformed likelihood, for example. Multiple SV candidates, of the plurality of SV candidates, meeting or exceeding a predefined likelihood threshold can be output from the first phase to result in multiple SV candidates provided to the second phase. A constraint may be provided as part of the SV candidate identification to ensure that at least two SV candidates are provided to the second phase.

In embodiments, the second phase identifies a plurality of star allele candidates and evaluates, for each star allele candidate of the plurality of star allele candidates, a cost of the star allele candidate. The cost of the star allele candidate could include a log transformed likelihood, for example. Each star allele call of the plurality of different star allele calls determined by applying the Bayesian graphical model can correspond to a star allele candidate identified by the second phase and a corresponding SV candidate identified by the first phase. The respective quality score for the star allele call of the plurality of different star allele calls determined by the applying the Bayesian graphical model can include a composite of (i) the cost of the star allele candidate identified by the second phase and (ii) the cost of the SV candidate identified by the first phase. For example, the composite can include a sum of the cost of the star allele candidate identified by the second phase and the cost of the SV candidate identified by the first phase.

Continuing with FIG. 10, the process provides (1006) a respective quality score for each star allele call of the plurality of different star allele calls. In embodiments, the Bayesian graphical model considers qualities and population frequencies of structural variants and star alleles in determining the respective quality score for each star allele call of the plurality of different star allele calls. In some examples, the respective quality score for each star allele call of the plurality of different star allele calls could include a log transformed likelihood converted to a posterior probability.

The process can also provide (1008), for each star allele call of the plurality of different star allele calls, one or more of (i) supporting variants for the star allele call, (ii) missing and/or masked Core Variants, or (iii) missing pharmacogenomic-related variants. Additionally, the process can rank (1010) the plurality of different star allele calls based on the respective quality score for each star allele call of the plurality of different star allele calls.

A sampling of aspects described herein is as follows:

A1. A computer-implemented method comprising: obtaining input genetic sequence variation data from a high-throughput genotyping platform based on a pharmacogenomic genotyping of a sample; applying a Bayesian graphical model to determine a plurality of different star allele calls corresponding to the sample; and providing a respective quality score for each star allele call of the plurality of different star allele calls.

A2. The method of A1, wherein the high-throughput genotyping platform comprises a microarray-based genotyping platform.

A3. The method of A1 or A2, wherein the input genetic sequence variation data comprises genotype data and copy number variant call data.

A4. The method of A3, wherein the genotype and copy number data comprises B-allele frequency (BAF) and log R ratio data.

A5. The method of A1, A2, A3, or A4, wherein the applying the Bayesian graphical model uses multi-solution integer programming to explore a model space of the Bayesian graphical model in (i) a first phase comprising structural variant (SV) candidate identification and (ii) a second phase comprising star allele candidate identification based on the SV candidate identification, to determine the plurality of different star allele calls.

A6. The method of A5, wherein the first phase identifies a plurality of SV candidates and evaluates, for each SV candidate of the plurality of SV candidates, a cost of the SV candidate.

A7. The method of A6, wherein the cost of the SV candidate comprises a log transformed likelihood.

A8. The method of A6 or A7, wherein multiple SV candidates, of the plurality of SV candidates, meeting or exceeding a predefined likelihood threshold are output from the first phase to result in multiple SV candidates provided to the second phase.

A9. The method of A5, A6, A7 or A8, wherein a constraint is provided as part of the SV candidate identification to ensure that at least two SV candidates are provided to the second phase.

A10. The method of A5, A6, A7, A8, or A9 wherein the second phase identifies a plurality of star allele candidates and evaluates, for each star allele candidate of the plurality of star allele candidates, a cost of the star allele candidate.

A11. The method of A10, wherein the cost of the star allele candidate comprises a log transformed likelihood.

A12. The method of A10 or A11, wherein each star allele call of the plurality of different star allele calls determined by applying the Bayesian graphical model corresponds to a star allele candidate identified by the second phase and a corresponding SV candidate identified by the first phase, and wherein the respective quality score for the star allele call of the plurality of different star allele calls determined by the applying the Bayesian graphical model comprises a composite of (i) the cost of the star allele candidate identified by the second phase and (ii) the cost of the SV candidate identified by the first phase.

A13. The method of A12, wherein the composite comprises a sum of the cost of the star allele candidate identified by the second phase and the cost of the SV candidate identified by the first phase.

A14. The method of A1, A2, A3, A4, A5, A6, A7, A8, A9, A10, A11, A12, or A13, wherein the Bayesian graphical model considers qualities and population frequencies of structural variants and star alleles in determining the respective quality score for each star allele call of the plurality of different star allele calls.

A15. The method of A1, A2, A3, A4, A5, A6, A7, A8, A9, A10, A11, A12, A13, or A14, further comprising, based on the respective quality score for each star allele call of the plurality of different star allele calls, ranking the plurality of different star allele calls.

A16. The method of A1, A2, A3, A4, A5, A6, A7, A8, A9, A10, A11, A12, A13, A14, or A15, wherein the respective quality score for each star allele call of the plurality of different star allele calls comprises a log transformed likelihood converted to a posterior probability.

A17. The method of A1, A2, A3, A4, A5, A6, A7, A8, A9, A10, A11, A12, A13, A14, A15 or A16, further comprising providing, for each star allele call of the plurality of different star allele calls, one or more of (i) supporting variants for the star allele call, (ii) missing and/or masked Core Variants, or (iii) missing pharmacogenomic-related variants.

B1. A computer system comprising: a memory; and a processor in communication with the memory, wherein the computer system is configured to perform a method comprising: obtaining input genetic sequence variation data from a high-throughput genotyping platform based on a pharmacogenomic genotyping of a sample; applying a Bayesian graphical model to determine a plurality of different star allele calls corresponding to the sample; and providing a respective quality score for each star allele call of the plurality of different star allele calls.

B2. The computer system of B1, wherein the high-throughput genotyping platform comprises a microarray-based genotyping platform.

B3. The computer system of B1 or B2, wherein the input genetic sequence variation data comprises genotype data and copy number variant call data.

B4. The computer system of B3, wherein the genotype and copy number data comprises B-allele frequency (BAF) and log R ratio data.

B5. The computer system of B1, B2, B3, or B4, wherein the applying the Bayesian graphical model uses multi-solution integer programming to explore a model space of the Bayesian graphical model in (i) a first phase comprising structural variant (SV) candidate identification and (ii) a second phase comprising star allele candidate identification based on the SV candidate identification, to determine the plurality of different star allele calls.

B6. The computer system of B5, wherein the first phase identifies a plurality of SV candidates and evaluates, for each SV candidate of the plurality of SV candidates, a cost of the SV candidate.

B7. The computer system of B6, wherein the cost of the SV candidate comprises a log transformed likelihood.

B8. The computer system of B6 or B7, wherein multiple SV candidates, of the plurality of SV candidates, meeting or exceeding a predefined likelihood threshold are output from the first phase to result in multiple SV candidates provided to the second phase.

B9. The computer system of B5, B6, B7 or B8, wherein a constraint is provided as part of the SV candidate identification to ensure that at least two SV candidates are provided to the second phase.

B10. The computer system of B5, B6, B7, B8, or B9 wherein the second phase identifies a plurality of star allele candidates and evaluates, for each star allele candidate of the plurality of star allele candidates, a cost of the star allele candidate.

B11. The computer system of B10, wherein the cost of the star allele candidate comprises a log transformed likelihood.

B12. The computer system of B10 or B11, wherein each star allele call of the plurality of different star allele calls determined by applying the Bayesian graphical model corresponds to a star allele candidate identified by the second phase and a corresponding SV candidate identified by the first phase, and wherein the respective quality score for the star allele call of the plurality of different star allele calls determined by the applying the Bayesian graphical model comprises a composite of (i) the cost of the star allele candidate identified by the second phase and (ii) the cost of the SV candidate identified by the first phase.

B13. The computer system of B12, wherein the composite comprises a sum of the cost of the star allele candidate identified by the second phase and the cost of the SV candidate identified by the first phase.

B14. The computer system of B1, B2, B3, B4, B5, B6, B7, B8, B9, B10, B11, B12, or B13, wherein the Bayesian graphical model considers qualities and population frequencies of structural variants and star alleles in determining the respective quality score for each star allele call of the plurality of different star allele calls.

B15. The computer system of B1, B2, B3, B4, B5, B6, B7, B8, B9, B10, B11, B12, B13, or B14, wherein the method further comprises, based on the respective quality score for each star allele call of the plurality of different star allele calls, ranking the plurality of different star allele calls.

B16. The computer system of B1, B2, B3, B4, B5, B6, B7, B8, B9, B10, B11, B12, B13, B14, or B15, wherein the respective quality score for each star allele call of the plurality of different star allele calls comprises a log transformed likelihood converted to a posterior probability.

B17. The computer system of B1, B2, B3, B4, B5, B6, B7, B8, B9, B10, B11, B12, B13, B14, B15 or B16, wherein the method further comprises providing, for each star allele call of the plurality of different star allele calls, one or more of (i) supporting variants for the star allele call, (ii) missing and/or masked Core Variants, or (iii) missing pharmacogenomic-related variants.

C1. A computer program product comprising: a computer readable storage medium readable by a processing circuit and storing instructions for execution by the processing circuit for performing a method comprising: obtaining input genetic sequence variation data from a high-throughput genotyping platform based on a pharmacogenomic genotyping of a sample; applying a Bayesian graphical model to determine a plurality of different star allele calls corresponding to the sample; and providing a respective quality score for each star allele call of the plurality of different star allele calls.

C2. The computer program product of C1, wherein the high-throughput genotyping platform comprises a microarray-based genotyping platform.

C3. The computer program product of C1 or C2, wherein the input genetic sequence variation data comprises genotype data and copy number variant call data.

C4. The computer program product of C3, wherein the genotype and copy number data comprises B-allele frequency (BAF) and log R ratio data.

C5. The computer program product of C1, C2, C3, or C4, wherein the applying the Bayesian graphical model uses multi-solution integer programming to explore a model space of the Bayesian graphical model in (i) a first phase comprising structural variant (SV) candidate identification and (ii) a second phase comprising star allele candidate identification based on the SV candidate identification, to determine the plurality of different star allele calls.

C6. The computer program product of C5, wherein the first phase identifies a plurality of SV candidates and evaluates, for each SV candidate of the plurality of SV candidates, a cost of the SV candidate.

C7. The computer program product of C6, wherein the cost of the SV candidate comprises a log transformed likelihood.

C8. The computer program product of C6 or C7, wherein multiple SV candidates, of the plurality of SV candidates, meeting or exceeding a predefined likelihood threshold are output from the first phase to result in multiple SV candidates provided to the second phase.

C9. The computer program product of C5, C6, C7 or C8, wherein a constraint is provided as part of the SV candidate identification to ensure that at least two SV candidates are provided to the second phase.

C10. The computer program product of C5, C6, C7, C8, or C9 wherein the second phase identifies a plurality of star allele candidates and evaluates, for each star allele candidate of the plurality of star allele candidates, a cost of the star allele candidate.

C11. The computer program product of C10, wherein the cost of the star allele candidate comprises a log transformed likelihood.

C12. The computer program product of C10 or C11, wherein each star allele call of the plurality of different star allele calls determined by applying the Bayesian graphical model corresponds to a star allele candidate identified by the second phase and a corresponding SV candidate identified by the first phase, and wherein the respective quality score for the star allele call of the plurality of different star allele calls determined by the applying the Bayesian graphical model comprises a composite of (i) the cost of the star allele candidate identified by the second phase and (ii) the cost of the SV candidate identified by the first phase.

C13. The computer program product of C12, wherein the composite comprises a sum of the cost of the star allele candidate identified by the second phase and the cost of the SV candidate identified by the first phase.

C14. The computer program product of C1, C2, C3, C4, C5, C6, C7, C8, C9, C10, C11, C12, or C13, wherein the Bayesian graphical model considers qualities and population frequencies of structural variants and star alleles in determining the respective quality score for each star allele call of the plurality of different star allele calls.

C15. The computer program product of C1, C2, C3, C4, C5, C6, C7, C8, C9, C10, C11, C12, C13, or C14, wherein the method further comprises, based on the respective quality score for each star allele call of the plurality of different star allele calls, ranking the plurality of different star allele calls.

C16. The computer program product of C1, C2, C3, C4, C5, C6, C7, C8, C9, C10, C11, C12, C13, C14, or C15, wherein the respective quality score for each star allele call of the plurality of different star allele calls comprises a log transformed likelihood converted to a posterior probability.

C17. The computer program product of C1, C2, C3, C4, C5, C6, C7, C8, C9, C10, C11, C12, C13, C14, C15, or C16, wherein the method further comprises providing, for each star allele call of the plurality of different star allele calls, one or more of (i) supporting variants for the star allele call, (ii) missing and/or masked Core Variants, or (iii) missing pharmacogenomic-related variants.

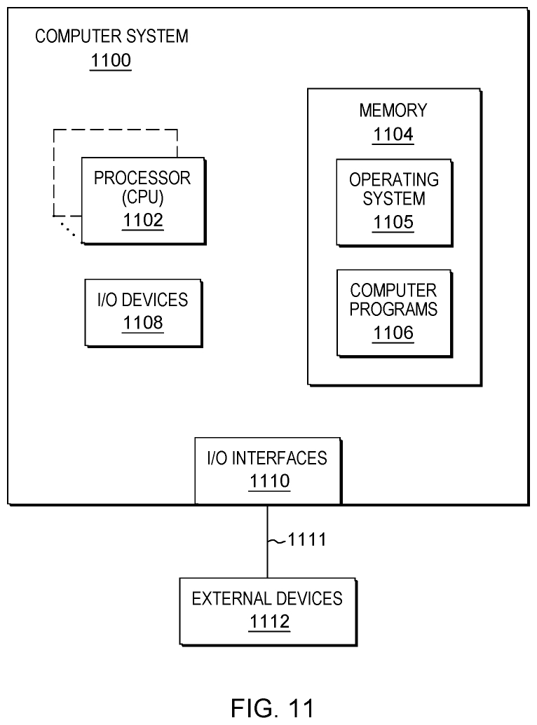

Processes described herein may be performed singly or collectively by one or more computer systems, such as one or more computer system(s) executing genomic analysis software to perform aspects described herein. FIG. 11 depicts an example of a computer system and associated devices to incorporate and/or use aspects described herein. A computer system may also be referred to herein as a data processing device/system, computing device/system/node, or simply a computer. The computer system may be based on one or more of various system architectures and/or instruction set architectures, such as those offered by Intel Corporation (Santa Clara, California, USA) as an example. FIG. 11 shows a computer system 1100 in communication with external device(s) 1112. Computer system 1100 includes one or more processor(s) 1102, for instance central processing unit(s) (CPUs). A processor can include functional components used in the execution of instructions, such as functional components to fetch program instructions from locations such as cache or main memory, decode program instructions, and execute program instructions, access memory for instruction execution, and write results of the executed instructions. A processor 1102 can also include register(s) to be used by one or more of the functional components. Computer system 1100 also includes memory 1104, input/output (I/O) devices 1108, and I/O interfaces 1110, which may be coupled to processor(s) 1102 and each other via one or more buses and/or other connections. Bus connections represent one or more of any of several types of bus structures, including a memory bus or memory controller, a peripheral bus, an accelerated graphics port, and a processor or local bus using any of a variety of bus architectures. By way of example, and not limitation, such architectures include the Industry Standard Architecture (ISA), the Micro Channel Architecture (MCA), the Enhanced ISA (EISA), the Video Electronics Standards Association (VESA) local bus, and the Peripheral Component Interconnect (PCI).

Memory 1104 can be or include main or system memory (e.g., Random Access Memory) used in the execution of program instructions, storage device(s) such as hard drive(s), flash media, or optical media as examples, and/or cache memory, as examples. Memory 1104 can include, for instance, a cache, such as a shared cache, which may be coupled to local caches (examples include L1 cache, L2 cache, etc.) of processor(s) 1102. Additionally, memory 1104 may be or include at least one computer program product having a set (e.g., at least one) of program modules, instructions, code or the like that is/are configured to carry out functions of embodiments described herein when executed by one or more processors.

Memory 1104 can store an operating system 1105 and other computer programs 1106, such as one or more computer programs/applications that execute to perform aspects described herein. Specifically, programs/applications can include computer readable program instructions that may be configured to carry out functions of embodiments of aspects described herein.

Examples of I/O devices 1108 include but are not limited to microphones, speakers, Global Positioning System (GPS) devices, cameras, lights, accelerometers, gyroscopes, magnetometers, sensor devices configured to sense light, proximity, heart rate, body and/or ambient temperature, blood pressure, and/or skin resistance, and activity monitors. An I/O device may be incorporated into the computer system as shown, though in some embodiments an I/O device may be regarded as an external device (1112) coupled to the computer system through one or more I/O interfaces 1110.

Computer system 1100 may communicate with one or more external devices 1112 via one or more I/O interfaces 1110. Example external devices include a keyboard, a pointing device, a display, and/or any other devices that enable a user to interact with computer system 1100. Other example external devices include any device that enables computer system 1100 to communicate with one or more other computing systems or peripheral devices such as a printer. A network interface/adapter is an example I/O interface that enables computer system 1100 to communicate with one or more networks, such as a local area network (LAN), a general wide area network (WAN), and/or a public network (e.g., the Internet), providing communication with other computing devices or systems, storage devices, or the like. Ethernet-based (such as Wi-Fi) interfaces and Bluetooth® adapters are just examples of the currently available types of network adapters used in computer systems (BLUETOOTH is a registered trademark of Bluetooth SIG, Inc., Kirkland, Washington, U.S.A.).

The communication between I/O interfaces 1110 and external devices 1112 can occur across wired and/or wireless communications link(s) 1111, such as Ethernet-based wired or wireless connections. Example wireless connections include cellular, Wi-Fi, Bluetooth®, proximity-based, near-field, or other types of wireless connections. More generally, communications link(s) 1111 may be any appropriate wireless and/or wired communication link(s) for communicating data.

Particular external device(s) 1112 may include one or more data storage devices, which may store one or more programs, one or more computer readable program instructions, and/or data, etc. Computer system 1100 may include and/or be coupled to and in communication with (e.g., as an external device of the computer system) removable/non-removable, volatile/non-volatile computer system storage media. For example, it may include and/or be coupled to a non-removable, non-volatile magnetic media (typically called a “hard drive”), a magnetic disk drive for reading from and writing to a removable, non-volatile magnetic disk (e.g., a “floppy disk”), and/or an optical disk drive for reading from or writing to a removable, non-volatile optical disk, such as a CD-ROM, DVD-ROM or other optical media.

Computer system 1100 may be operational with numerous other general purpose or special purpose computing system environments or configurations. Computer system 1100 may take any of various forms, well-known examples of which include, but are not limited to, personal computer (PC) system(s), server computer system(s), such as messaging server(s), thin client(s), thick client(s), workstation(s), laptop(s), handheld device(s), mobile device(s)/computer(s) such as smartphone(s), tablet(s), and wearable device(s), multiprocessor system(s), microprocessor-based system(s), telephony device(s), network appliance(s) (such as edge appliance(s)), virtualization device(s), storage controller(s), set top box(es), programmable consumer electronic(s), network PC(s), minicomputer system(s), mainframe computer system(s), and distributed cloud computing environment(s) that include any of the above systems or devices, and the like.

Aspects of the present invention may be a system, a method, and/or a computer program product, any of which may be configured to perform or facilitate aspects described herein.

In some embodiments, aspects of the present invention may take the form of a computer program product, which may be embodied as computer readable medium(s). A computer readable medium may be a tangible storage device/medium having computer readable program code/instructions stored thereon. Example computer readable medium(s) include, but are not limited to, electronic, magnetic, optical, or semiconductor storage devices or systems, or any combination of the foregoing. Example embodiments of a computer readable medium include a hard drive or other mass-storage device, an electrical connection having wires, random access memory (RAM), read-only memory (ROM), erasable-programmable read-only memory such as EPROM or flash memory, an optical fiber, a portable computer disk/diskette, such as a compact disc read-only memory (CD-ROM) or Digital Versatile Disc (DVD), an optical storage device, a magnetic storage device, or any combination of the foregoing. The computer readable medium may be readable by a processor, processing unit, or the like, to obtain data (e.g., instructions) from the medium for execution. In a particular example, a computer program product is or includes one or more computer readable media that includes/stores computer readable program code to provide and facilitate one or more aspects described herein.

As noted, program instruction contained or stored in/on a computer readable medium can be obtained and executed by any of various suitable components such as a processor of a computer system to cause the computer system to behave and function in a particular manner. Such program instructions for carrying out operations to perform, achieve, or facilitate aspects described herein may be written in, or compiled from code written in, any desired programming language. In some embodiments, such programming language includes object-oriented and/or procedural programming languages such as C, C++, C#, Java, etc.

Program code can include one or more program instructions obtained for execution by one or more processors. Computer program instructions may be provided to one or more processors of, e.g., one or more computer systems, to produce a machine, such that the program instructions, when executed by the one or more processors, perform, achieve, or facilitate aspects of the present invention, such as actions or functions described in flowcharts and/or block diagrams described herein. Thus, each block, or combinations of blocks, of the flowchart illustrations and/or block diagrams depicted and described herein can be implemented, in some embodiments, by computer program instructions.

Although various embodiments are described above, these are only examples.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components and/or groups thereof.

The corresponding structures, materials, acts, and equivalents of all means or step plus function elements in the claims below, if any, are intended to include any structure, material, or act for performing the function in combination with other claimed elements as specifically claimed. The description of one or more embodiments has been presented for purposes of illustration and description, but is not intended to be exhaustive or limited to in the form disclosed. Many modifications and variations will be apparent to those of ordinary skill in the art. The embodiment was chosen and described in order to best explain various aspects and the practical application, and to enable others of ordinary skill in the art to understand various embodiments with various modifications as are suited to the particular use contemplated.

What is claimed is:

1. A computer-implemented method comprising:
obtaining input genetic sequence variation data from a high-throughput genotyping platform based on a pharmacogenomic genotyping of a sample;
applying a Bayesian graphical model to determine a plurality of different star allele calls corresponding to the sample; and
providing a respective quality score for each star allele call of the plurality of different star allele calls.

2. The method of claim 1, wherein the high-throughput genotyping platform comprises a microarray-based genotyping platform.

3. The method of claim 1, wherein the input genetic sequence variation data comprises genotype data and copy number variant call data.

4. The method of claim 3, wherein the genotype and copy number data comprises B-allele frequency (BAF) and log R ratio data.

5. The method of claim 1, wherein the applying the Bayesian graphical model uses multi-solution integer programming to explore a model space of the Bayesian graphical model in (i) a first phase comprising structural variant (SV) candidate identification and (ii) a second phase comprising star allele candidate identification based on the SV candidate identification, to determine the plurality of different star allele calls.

6. The method of claim 5, wherein the first phase identifies a plurality of SV candidates and evaluates, for each SV candidate of the plurality of SV candidates, a cost of the SV candidate.

7. The method of claim 6, wherein multiple SV candidates, of the plurality of SV candidates, meeting or exceeding a predefined likelihood threshold are output from the first phase to result in multiple SV candidates provided to the second phase.

8. The method of claim 5, wherein a constraint is provided as part of the SV candidate identification to ensure that at least two SV candidates are provided to the second phase.

9. The method of claim 5, wherein the second phase identifies a plurality of star allele candidates and evaluates, for each star allele candidate of the plurality of star allele candidates, a cost of the star allele candidate.

10. The method of claim 9, wherein at least one of (i) the cost of an SV candidate of the plurality of SV candidates or (ii) the cost of a star allele candidate of the plurality of star allele candidates comprises a respective log transformed likelihood.

11. The method of claim 9, wherein each star allele call of the plurality of different star allele calls determined by applying the Bayesian graphical model corresponds to a star allele candidate identified by the second phase and a corresponding SV candidate identified by the first phase, and wherein the respective quality score for the star allele call of the plurality of different star allele calls determined by the applying the Bayesian graphical model comprises a composite of (i) the cost of the star allele candidate identified by the second phase and (ii) the cost of the SV candidate identified by the first phase.

12. The method of claim 11, wherein the composite comprises a sum of the cost of the star allele candidate identified by the second phase and the cost of the SV candidate identified by the first phase.

13. The method of claim 1, wherein the Bayesian graphical model considers qualities and population frequencies of structural variants and star alleles in determining the respective quality score for each star allele call of the plurality of different star allele calls.

14. The method of claim 1, further comprising, based on the respective quality score for each star allele call of the plurality of different star allele calls, ranking the plurality of different star allele calls.

15. The method of claim 1, wherein the respective quality score for each star allele call of the plurality of different star allele calls comprises a log transformed likelihood converted to a posterior probability.

16. The method of claim 1, further comprising providing, for each star allele call of the plurality of different star allele calls, one or more of (i) supporting variants for the star allele call, (ii) missing and/or masked Core Variants, or (iii) missing pharmacogenomic-related variants.

17. A computer system comprising:
a memory; and
a processor in communication with the memory, wherein the computer system is configured to perform a method comprising:
obtaining input genetic sequence variation data from a high-throughput genotyping platform based on a pharmacogenomic genotyping of a sample;
applying a Bayesian graphical model to determine a plurality of different star allele calls corresponding to the sample; and
providing a respective quality score for each star allele call of the plurality of different star allele calls.

18. The computer system of claim 17, wherein the applying the Bayesian graphical model uses multi-solution integer programming to explore a model space of the Bayesian graphical model in (i) a first phase comprising structural variant (SV) candidate identification and (ii) a second phase comprising star allele candidate identification based on the SV candidate identification, to determine the plurality of different star allele calls, wherein the first phase identifies a plurality of SV candidates and evaluates, for each SV candidate of the plurality of SV candidates, a cost of the SV candidate, wherein the second phase identifies a plurality of star allele candidates and evaluates, for each star allele candidate of the plurality of star allele candidates, a cost of the star allele candidate, wherein each star allele call of the plurality of different star allele calls determined by applying the Bayesian graphical model corresponds to a star allele candidate identified by the second phase and a corresponding SV candidate identified by the first phase, and wherein the respective quality score for the star allele call of the plurality of different star allele calls determined by the applying the Bayesian graphical model comprises a composite of (i) the cost of the star allele candidate identified by the second phase and (ii) the cost of the SV candidate identified by the first phase.

19. A computer program product comprising:
a computer readable storage medium readable by a processing circuit and storing instructions for execution by the processing circuit for performing a method comprising:
obtaining input genetic sequence variation data from a high-throughput genotyping platform based on a pharmacogenomic genotyping of a sample;
applying a Bayesian graphical model to determine a plurality of different star allele calls corresponding to the sample; and
providing a respective quality score for each star allele call of the plurality of different star allele calls.

20. The computer program product of claim 19, wherein the applying the Bayesian graphical model uses multi-solution integer programming to explore a model space of the Bayesian graphical model in (i) a first phase comprising structural variant (SV) candidate identification and (ii) a second phase comprising star allele candidate identification based on the SV candidate identification, to determine the plurality of different star allele calls, wherein the first phase identifies a plurality of SV candidates and evaluates, for each SV candidate of the plurality of SV candidates, a cost of the SV candidate, wherein the second phase identifies a plurality of star allele candidates and evaluates, for each star allele candidate of the plurality of star allele candidates, a cost of the star allele candidate, wherein each star allele call of the plurality of different star allele calls determined by applying the Bayesian graphical model corresponds to a star allele candidate identified by the second phase and a corresponding SV candidate identified by the first phase, and wherein the respective quality score for the star allele call of the plurality of different star allele calls determined by the applying the Bayesian graphical model comprises a composite of (i) the cost of the star allele candidate identified by the second phase and (ii) the cost of the SV candidate identified by the first phase.
